(19) United States
(12) Patent Application Publication
(10) Pub. No.: US 2009/0060915 A1
(43) Pub. Date: Mar. 5, 2009

(54) POLYPEPTIDE, VACCINE AND USE THEREOF

(75) Inventor: Lars Holmgren, Bromma (SE)

Correspondence Address: FISH & RICHARDSON P.C., PO BOX 1022, MINNEAPOLIS, MN, 55440-1022, US

(73) Assignee: BIOINVENT INTERNATIONAL AB, Lund (SE)

(21) Appl. No.: 11/913,041

(22) PCT Filed: Apr. 28, 2006
(86) PCT No.: PCT/EP2006/004320

§ 371 Date: Apr. 30, 2008

(30) Foreign Application Priority Data
Apr. 29, 2005 (GB) 0508726.7

(51) Int. Cl. A61K 39/395; C07K 14/00; C07K 16/18; A61K 39/00; A61P 31/00; A61K 38/00; A61K 31/7088

(52) U.S. Cl. 424/138.1; 530/350; 530/387.9; 514/12; 514/44; 424/185.1

(57) ABSTRACT

The present invention relates to prophylactic or therapeutic treatments for hindering blood vessel formation (for example, angiogenesis) for example for hindering tumour growth. In particular, the invention relates to a vaccine or medicament comprising a whole angiomotin molecule or a fragment thereof which may be used to generate immune responses to angiomotin. The invention also provides antibodies specific for a whole angiomotin molecule or a fragment thereof for use in prophylactic or therapeutic treatments.

The present invention relates to prophylactic or therapeutic treatments for hindering blood vessel formation, for example for hindering tumour growth, retinal disease, atherosclerosis, endometriosis, rheumatoid arthritis and inflammatory conditions.

One form of prophylactic or therapeutic treatment is vaccination. A vaccine is a preparation derived from a disease-causing agent or its components which is administered to stimulate an immune response that will protect a person from illness due to that agent. A therapeutic (treatment) vaccine is given after onset of the disease and is intended to reduce or arrest disease progression. A preventive (prophylactic) vaccine is intended to prevent initial disease onset. Agents used in vaccines may, for example, be whole-killed (inactive), live-attenuated (weakened) pathogenic organisms or artificially manufactured.

Vaccines mediate their effect by stimulating the immune system of the host to specifically generate antibodies and/or immune cells (cytotoxic T-cells) against the principal target. These targets are known as “antigens”. Stimulation of immune responses against the target antigen results in the immune-mediated destruction and elimination of the disease agent residing in the body of the immunized host. Once stimulated, the immune system also maintains surveillance against subsequent infection or development of disease by the targeted agent. Thus, vaccines are an effective way of controlling existing disease as well as inhibiting its exacerbation or recurrence in the future.

There are a variety of methods for vaccinating against a target antigen. Some of these include injecting a whole protein; a synthetic peptide corresponding to a fragment of the protein; and/or DNA/RNA sequences encoding the protein. Delivery vehicles for delivering DNA and RNA include altered viruses expressing the gene, naked DNA/RNA or recombinant plasmids that express the antigen in conjunction with other immunostimulatory agents such as cytokines or growth factors.

As an alternative to vaccination, prophylactic or therapeutic treatment can be achieved by treating a host with a therapeutic antibody that has been generated outside the host and which is specific for an antigen of interest.

Methods for generating, isolating and using antibodies for a desired antigen or epitope are well known to those skilled in the relevant art. For example, an antibody may be raised in a suitable host animal (such as, for example, a mouse, rabbit or goat) using standard methods known in the art and either used as crude antisera or purified, for example by affinity purification. An antibody of desired specificity may alternatively be generated using well-known molecular biology methods, including selection from a molecular library of recombinant antibodies, or grafting or shuffling of complementarity-determining regions (CDRs) onto appropriate framework regions. Human antibodies may be selected from recombinant libraries and/or generated by grafting CDRs from non-human antibodies onto human framework regions using well-known molecular biology techniques. Such antibodies can be used as therapeutic treatments—for example, a medicament comprising therapeutic antibodies may be introduced into a subject to modulate the immune response of that subject. For example, a therapeutic antibody specific for an antigen in the subject will stimulate an immune response to that antigen, thereby inducing and/or promoting an immune response and aiding recovery.

Vasculogenesis is the differentiation of stem cells into endothelial cells which then form blood vessels. Angiogenesis is the formation of blood vessels from pre-existing ones. Terms such as blood vessel formation, neovascularisation and vascularisation cover both vasculogenesis and angiogenesis.

Angiogenesis (an example of blood vessel formation) is the formation of new capillary blood vessels by a process of sprouting from pre-existing vessels and occurs during development as well as in a number of physiological and pathological settings (Folkman, 1995, Nature Medicine, 1:27-31). Formation of new blood vessels by the process of angiogenesis involves a complex series of events including endothelial cell proliferation, migration, interaction and adhesion to form cords and tubes, and finally maturation. Physiologically, angiogenesis is necessary for tissue growth, wound healing, and female reproductive function and is a component of pathological processes such as retinal disease, atherosclerosis, endometriosis, rheumatoid arthritis and inflammatory conditions. However, much of the longstanding interest in angiogenesis comes from the notion that for solid tumours to grow beyond a critical size, they must recruit endothelial cells from the surrounding stroma to form their own endogenous microcirculation. In order to promote neo-vascularisation, tumours release variety of factors that stimulate proliferation and migration of endothelial cells. Such factors include vascular endothelial cell growth factor (VEGF) and basic fibroblast growth factor (bFGF), interleukin-8 (IL-8) placental growth factor, and thymidine phosphorylase (platelet-derived endothelial cell growth factor; Relf et al., 1997, Cancer Research, 57:963-9). Therefore, much effort has been dedicated to finding molecules that interfere with these signalling pathways and thereby block tumour angiogenesis.

Targeting angiogenesis has potentially several advantages compared to traditional oncolytic therapy. The most prominent of these is that: all solid tumours are angiogenesis-dependent; the target endothelial cells are readily accessible for therapy; and they are genomically stable and less prone to generate resistance to therapy. One of the obvious disadvantages of targeting a cancer cell-expressed protein is the genetic variability and a large selection pressure due to rapid cell growth and division, which often renders such drugs ineffective due to resistance mechanisms and the onset and use of alternative pathways.

Angiogenesis inhibition is also showing early promise with diabetic retinopathy and macular degeneration, which both result from an overgrowth of ocular blood vessels. In these disorders, the vessels interfere with normal structures in the eye, or block light from reaching the back of the eye. The new blood vessels are themselves the primary pathology, and stopping blood vessel growth could prevent blindness.

A large number of naturally-occurring angiogenic inhibitors have been identified such as angiostatin (a 38 kDa proteolytic fragment of plasminogen), anti-angiogenic anti-thrombin III, endostatin (collagen XVIII fragment), interferon alpha/beta/gamma, prolactin 16 kDa fragment and thrombospondin-1 (TSP-1) which show varying degrees of effect in in vitro and in vivo models. These inhibitors target endothelial cells and inhibit angiogenesis. The observed inhibition of, for example, angiostatin, is independent of which angiogenic factor the endothelial cells are stimulated by (Eriksson et al., 2003 FEBS Letts., 536:19-24). This is in contrast to agents such as antibodies that bind to VEGF or low molecular compounds that inhibit VEGF-receptor kinase activity. Most tumours express a variety of angiogenic factors indicating that targeting one single angiogenesis pathway is not enough for inhibiting tumour expansion. Thus, therapies that target directly the endothelial cells have a potential to circumvent the problem of angiogenesis being controlled by a plurality of tumor-derived factors. However, on the other hand, such therapies have to deal with the problem of being able to target specifically endothelial cells that are involved in the process of neo-vascularisation while sparing mature blood vessels.

An 80 kDa molecule named angiomotin (“p80-angiomotin”) was identified by its ability to bind to the angiogenesis inhibitor, angiostatin (Troyanovsky et al., 2001, J. Cell. Biol., 152:1247-1254; WO 99/66038). p80-angiomotin belongs to a new protein family with two additional members, angiomotin-like protein 1 (Amot1) and 2 (Amot2). These proteins are characterised by conserved coiled-coil domains and C-terminal PDZ binding motifs (Nishimura et al., 2002, J. Biol. Chem., 277:5583-5587; Bratt et al., 2002, Gene, 298:69-77). p80-angiomotin differs from its related proteins as it contains an extracellular angiostatin-binding domain. P80-angiomotin is primarily expressed in endothelial cells and mediates the inhibitory effect of angiostatin on endothelial cell migration and tube formation in vitro (Troyanovsky et al., 2001, J. Cell. Biol., 152:1247-1254). Expression of p80-angiomotin in mouse aortic endothelial (MAE) cells increases the migratory response to chemotactic factors (Troyanovsky et al., 2001, J. Cell. Biol., 152:1247-1254). A role in migration is further emphasised by the findings from p80-angiomotin knock-out experiments in mice in which approximately 70% of knock-out mice died during embryonic day 7-8 due to a migratory defect in the anterior visceral endoderm (Shimono and Behringer, 2003, Current Biology, 13:613-7).

Real-time PCR analysis of the expression pattern of p80-angiomotin in primary cells as well as in cell-lines have shown that angiomotin is predominantly expressed in endothelial cells. In vivo mapping of p80-angiomotin has revealed expression in angiogenic tissues such as the human placenta as well as tumour tissues. These data suggest that angiomotin is upregulated in endothelial cells during angiogenesis. WO 99/66038 discusses p80-angiomotin and its use as, for example, a drug screening target.

A novel splice isoform of p80-angiomotin that is 130 kDa (named “p130-angiomotin”) has recently been identified. This larger isoform contains an extended N-terminal domain and plays a role in angiogenesis that is distinct from, but related to, p80-angiomotin.

The invention provides the use of vaccines corresponding to the whole p130-angiomotin molecule or fragments thereof, for generating immune responses which hinder the formation of blood vessels (angiogenesis). The invention also provides vaccination with a p130-angiomotin molecule and methods of using the vaccine to prevent formation of blood vessels that are critical for tumour growth, as well as other diseases produced or exacerbated by neo-angiogenesis.

The invention further provides therapeutic antibodies, and fragments thereof, specific for p130-angiomotin, for use as a medicament to hinder the formation of blood vessels (angiogenesis) and/or prevent formation of blood vessels that are critical for tumour growth, as well as other diseases produced or exacerbated by neo-angiogenesis.

A first aspect of the invention provides an isolated or recombinant p130-angiomotin polypeptide for modulating angiogenesis and/or tumour formation.

A variety of methods for isolating polypeptides are known to those skilled in the art. For example, polypeptides may be isolated from a cell expressing the protein by use of affinity purification. Methods for producing recombinant polypeptides are also well known and include expression in bacterial, yeast, insect and mammalian cell systems. Methods for isolation of polypeptides and expression of recombinant polypeptides are discussed, for example, in Sambrook et al., 2001, Molecular Cloning: A Laboratory Manual (3rd Edition).

By “polypeptide” we include a chain of about ten or more amino acids (or derivatives thereof) linked to one another by peptide bonds. Polypeptides of the invention may be modified by the addition of one or more chemical group (such as, for example, phosphate groups and farnesyl groups). The present invention also includes polypeptides with or without glycosylation. Polypeptides expressed in yeast or mammalian expression systems may be similar or slightly different in molecular weight and glycosylation pattern to the native molecules, depending upon the expression system. For instance, expression of DNA encoding polypeptides in bacteria such as E. coli typically provides non-glycosylated molecules. N-glycosylation sites of eukaryotic proteins are characterized by the amino acid triplet Asn-A.sub.1-Z (where A.sub.1 is any amino acid except Proline, and Z is Serine or Threonine). Variants of polypeptides having inactivated N-glycosylation sites can be produced by techniques known to those of ordinary skill in the art, such as oligonucleotide synthesis and ligation or site-specific mutagenesis techniques, and are within the scope of this invention. Alternatively, N-linked glycosylation sites can be added to a polypeptide of the invention.

By “p130-angiomotin” we include any full-length naturally-occurring 130 kDa angiomotin polypeptide (for example, the p130-angiomotin polypeptide described by the present inventors in the Examples) or any variant thereof which retains antigenic cross-reactivity with the naturally-occurring p130-angiomotin polypeptide or fragment thereof.

Preferably, the polypeptide is an isolated or recombinant mammalian p130-angiomotin; more preferably, an isolated or recombinant human p130-angiomotin.

More preferably, the polypeptide comprises:
(i) the sequence of SEQ ID NO:1; or
(ii) a sequence which has at least 80% and/or at least 90% and/or at least 95% and/or at least 98% identity to SEQ ID NO:1 and provides a functional polypeptide; or
(iii) a functional fragment of SEQ ID NO:1 or the sequence of (ii).
   and wherein the polypeptide is not p80-angiomotin or a fragment of p80-angiomotin.

The term “angiomotin” is well known to those skilled in the art, and includes a polypeptide which: has coiled-coil and C-terminal PDZ binding domains; is considered to be a cell surface-associated protein; and is considered to bind to angiostatin and to mediate inhibitory effects of angiostatin on endothelial cell migration and tube formation. Examples of naturally-occurring angiomotin polypeptides are given in the following: Troyanovsky et al., 2001, J. Cell Biol., 152:1247-1254; WO 99/66038; Levchenko et al., 2003, J. Cell Sci., 116:3803-3810; Bratt et al., 2002, Gene, 298:69-77; GenBank accession No: NP_{—}573572 (human).

However, p130-angiomotin polypeptides and/or fragments thereof that are identical to p80-angiomotin polypeptides and fragments thereof are not considered to be polypeptides of the invention.

By “a functional polypeptide” and/or “a functional fragment of SEQ ID NO:1” we include any fragment of SEQ ID NO:1 which exhibits some or all of the cellular function(s) of SEQ ID NO:1. Preferably, the functional fragment of SEQ ID NO:1 is the N-terminal region of p130-angiomotin that does not have similarity to p80-angiomotin, and more preferably is a fragment comprising or consisting of amino acid residues 1 to 409 of SEQ ID NO:1. As shown in the Examples, the N-terminus of p130-angiomotin is capable of inducing stress fibres and altering cell shape, both of which are functions of full-length p130-angiomotin involved in promoting angiogenesis.

A second aspect of the invention provides an antibody or a fragment thereof which is capable of binding to a p130-angiomotin polypeptide of the invention.

Antibodies comprise two identical polypeptides of Mᵣ 50,000-70,000 (termed “heavy chains”) that are linked together by a disulphide bond, each of which is linked to one of an identical pair of polypeptides of Mᵣ 25,000 (termed “light chains”). There is considerable sequence variability between individual N-termini of heavy chains of different antibody molecules and between individual light chains of different antibody molecules and these regions have hence been termed “variable domains”. Conversely, there is considerable sequence similarity between individual C-termini of heavy chains of different antibody molecules and between individual light chains of different antibody molecules and these regions have hence been termed “constant domains”.

The antigen-binding site is formed from hyper-variable regions in the variable domains of a pair of heavy and light chains. The hyper-variable regions are also known as complementarity-determining regions (CDRs) and determine the specificity of the antibody for a ligand. The variable domains of the heavy chain (V_{H}) and light chain (V_{L}) typically comprise three CDRs, each of which is flanked by sequence with less variation, which are known as framework regions (FRs).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al., 1984, Proc. Natl. Acad. Sci. USA, 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al., 1988, Science, 240:1041). Fv molecules (Skerra et al., 1988, Science, 240, 1038); single-chain Fv (ScFv) molecules where the V_{H} and V_{L} partner domains are linked via a flexible oligopeptide (Bird et al., 1988, Science 242:423; Huston et al., 1988, Proc. Natl. Acad. Sci. USA, 85:5879) and single domain antibodies (dAbs) comprising isolated V domains (Ward et al., 1989 Nature 341, 544). A general review of the techniques involved in the synthesis of antibody fragments which retain their specific binding sites is to be found in Winter et al., 1991, Nature, 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from Escherichia coli (E. coli), thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site.

Methods for generating, isolating and using antibodies for a desired antigen or epitope are well known to those skilled in the relevant art. For example, an antibody may be raised in a suitable host animal (such as, for example, a mouse, rabbit or goat) using standard methods known in the art and either used as crude antisera or purified, for example by affinity purification. An antibody of desired specificity may alternatively be generated using well-known molecular biology methods, including selection from a molecular library of recombinant antibodies, or grafting or shuffling of complementarity-determining regions (CDRs) onto appropriate framework regions. Human antibodies may be selected from recombinant libraries and/or generated by grafting CDRs from non-human antibodies onto human framework regions using well-known molecular biology techniques.

Preferably, the antibody or fragment is capable of binding to a region of a p130-angiomotin polypeptide defined by residue 1 to residue 409 of SEQ ID NO:1 but that is not capable of binding to a p80-angiomotin polypeptide. Residues 1-409 of SEQ ID NO:1 correspond to the N-terminus of p130-angiomotin which does not have identity with the polypeptide sequence of p80-angiomotin.

Preferably, the antibody or fragment is capable of binding to a region of a p130-angiomotin polypeptide defined by residue 410 to residue 1084 of SEQ ID NO:1 but that is not capable of binding to a p80-angiomotin polypeptide. Residues 410-1084 of SEQ ID NO:1 correspond to the C-terminus of p130-angiomotin which has similarity to the polypeptide sequence of p80-angiomotin.

Preferably, the antibody or fragment is capable of binding to a region of a p130-angiomotin polypeptide defined by residue 871 to residue 1013 of SEQ ID NO:1 but that is not capable of binding to a p80-angiomotin polypeptide. Residues 871-1013 of SEQ ID NO:1 correspond to the angiostatin-binding domain of p130-angiomotin which has similarity to the polypeptide sequence of the angiostatin-binding domain of p80-angiomotin.

Preferably, the antibody is a human antibody.

Antibodies capable of binding to p80-angiomotin polypeptides and fragments thereof may not be considered to be antibodies of the invention.

A third aspect of the invention provides a use of an angiomotin polypeptide of the invention and/or its encoding polynucleotide (and/or antisense polynucleotide) for generating antibodies capable of binding to a p130-angiomotin polypeptide of the invention.

Methods for generating, isolating and using antibodies for a desired antigen or epitope (for example, a p130-angiomotin polypeptide of the invention) are well known to those skilled in the art and include those methods discussed above.

A fourth aspect of the invention provides an angiomotin polypeptide of the invention and/or its encoding polynucleotide (and/or antisense polynucleotide) and/or an antibody or fragment of the invention for use in medicine.

By “polynucleotide” we include single-stranded and/or double-stranded molecules of DNA (deoxyribonucleic acid) and/or RNA (ribonucleic acid) and derivatives thereof. By “encoding polynucleotide” we include a polynucleotide the sequence of which that may be translated to form a desired polypeptide. By “antisense polynucleotide” we include a polynucleotide that is complementary to the coding strand of an encoding polynucleotide.

The present inventors have demonstrated that p130-angiomotin and p80-angiomotin play a role in modulating vasculogenesis/angiogenesis. In particular, these polypeptides are involved in promoting vasculogenesis/angiogenesis by modulating cell migration, cell size and tube formation. Angiomotin polypeptides of the invention and/or its encoding polynucleotide (and/or antisense polynucleotide) and/or an antibody or fragment of the invention could therefore be used in medicine.

There are a number of medical conditions characterised by a loss of regulation control of the molecular processes controlling vasculogenesis and/or angiogenesis. For example, vasculogenesis/angiogenesis-related diseases include cancer (particularly solid tumours), haemangioma, ocular neovascularisation, diabetic retinopathy, macular degeneration, rheumatoid arthritis, inflammatory conditions (such as psoriasis, chronic inflammation of the intestines, asthma) and endometriosis.

The inventors have previously shown that an angiomotin molecule (in particular, p80-angiomotin) can be used effectively as a therapeutic and/or prophylactic treatment against vasculogenesis and/or angiogenesis in PCT/EP2004/014573, the disclosure of which is incorporated herein by reference.

A fifth aspect of the invention provides a use of an angiomotin polypeptide of the invention and/or its encoding polynucleotide and/or an antibody or fragment of the invention, in the manufacture of a medicament for modulating angiogenesis and/or tumour formation.

Methods for formulating polypeptides, polynucleotides and antibodies into medicaments, pharmaceutical compositions and vaccines are well-known to those in the relevant art. Preferred formulations of medicaments, pharmaceutical compositions and vaccines comprising the polypeptides, polynucleotides and antibodies of the invention are described in the Examples.

Preferably, the medicament prevents and/or reduces angiogenesis and/or tumour formation.

A number of well-known methods can be used for detecting and/or measuring angiogenesis and/or tumour formation and it will be understood that these may be used to determine whether a medicament prevents and/or reduces angiogenesis and/or tumour formation. The efficacy of vaccination can be measured, for example, by: analysing antibody titres using Enzyme Linked Immunosorbent Assays (ELISAs); indirectly measuring T cell activation by gamma-interferon production using Enzyme Linked Immunospot (ELISPOT) assays. The level of circulating endothelial cells may also be quantified and used as a surrogate marker for successful vaccination. The “level” of angiogenesis may be measured by determining tube formation and the presence of endothelial cells, as described in the Examples and in Troyanovsky et al., 2001, J. Cell. Biol., 152:1247-1254).

Tumours may be detected and/or measured for example by determining tumour size and volume using a micrometer. Neo-vascularisation in a tumour may be assessed by various known assays, including the “Matrigel-plug” assay (as described in, for example, London et al., 2003, Cancer Gene Ther., 10:823-832) or imaging-based assays such as the skin-flap window-chamber model.

A sixth aspect of the invention provides a use of an angiomotin polypeptide of the invention and/or its encoding polynucleotide (and/or antisense polynucleotide) and/or an antibody or fragment of the invention, in the manufacture of a medicament for treating a subject with an angiogenesis-related disease or disorder.

A seventh aspect of the invention provides a use of an angiomotin polypeptide of the invention and/or its encoding polynucleotide (and/or antisense polynucleotide) and/or an antibody or fragment of the invention in the manufacture of a vaccine for vaccinating a subject with, or at risk of, angiogenesis and/or tumour formation and/or an angiogenesis-related disease or disorder.

Preferably, the angiogenesis-related disease or disorder is cancer, a solid tumour, haemangioma, ocular neovascularisation, diabetic retinothapy, macular degeneration, rheumatoid arthritis, inflammatory conditions, psoriasis, chronic inflammation of the intestines, asthma or endometriosis.

A patient at risk of vasculogenesis/angiogenesis-related disease may be a patient at risk of cancer, particularly at risk of a solid tumour, for example a patient with a genetic predisposition to a form of cancer leading to a solid tumour, or a patient at environmental risk of a solid tumour. For example, a patient at risk of cancer may be a person with familial history of cancer who present polyps in the colon; or a woman known to be infected with a strain of human papilloma virus linked with cervical cancer and/or presenting pap smears demonstrating the earliest changes of cervical cancer.

Preferably, the subject to be treated is human, for example a human with or at risk of an angiogenesis-associated disease or condition (as defined above). Alternatively, the recipient may be an animal with or at risk of such a condition, for example a domesticated animal (e.g. a cat or dog) or animal important in agriculture, for example cattle, sheep, goats, or poultry.

The medicament or treatment may be a prophylactic treatment or a therapeutic treatment.

Methods for generating, isolating and using antibodies for a desired antigen or epitope are well known to those skilled in the relevant art and are discussed above. Such antibodies may be used in therapy—for example, a medicament comprising therapeutic antibodies may be introduced into a subject to modulate the immune response of that subject. For example, a therapeutic antibody specific for an antigen in the subject will stimulate an immune response to that antigen, thereby inducing and/or promoting an immune response and aiding recovery. Methods for administering therapeutic antibodies to a patient in need thereof are well known in the art. It will be understood that an antibody of the present invention may be used as a therapeutic antibody to modulate the immune response in a subject to p130-angiomotin and preferably inhibit and/or reduce angiogenesis in that subject.

Vaccination may be performed by administering the angiomotin polypeptide or polynucleotide of the invention to the subject; or by exposing immune cells of the subject to the angiomotin polypeptide or polynucleotide of the invention outside the subject's body, followed by returning the exposed immune cells (and/or their progeny) to the subject.

The scope of vaccinating with the polypeptide or polynucleotide of the invention as the immunogen is not restricted and encompasses protein, peptide and gene-based vaccination strategies. For example, the vaccine may comprise the polypeptide or polynucleotide of the invention or an immunostimulatory derivative or fragment thereof. It may be desirable to administer both protein or peptide-based and polynucleotide/gene-based vaccine components to a subject, either at the same time or sequentially. This may promote a stronger, broader or more balanced immune response.

Derivatives include, but are not limited to: (A) analogues of the polypeptide of the invention, where the amino acid sequence of the native protein is modified at one or more amino acid positions, to increase the immunogenicity of the molecule; (B) chemical modification of one or more amino acids such as substitution of one or more chemical groups naturally occurring on the said amino acid with an artificial chemical group to increase the immunogenicity of the resultant peptide; (C) conjugation of a peptide corresponding to a fragment of the polypeptide of the invention with an immunogenic protein (e.g. keyhole limpet hemocyanin—KLH) or a hapten (i.e. a small chemical molecule such as dinitrophenol—DNP).

The vaccine is considered to be capable of breaking tolerance and generating an immune response against endogenous p130-angiomotin in the recipient. For example a vaccine effective against blood vessel formation in a human may comprise an effective amount of a human p130-angiomotin molecule and/or its encoding polynucleotide; the p130-angiomotin molecule or encoded p130-angiomotin polypeptide may be full length angiomotin or a fragment thereof that promotes an immune response against epitope(s) that are present and accessible in endogenous p130-angiomotin.

The vaccine may further comprise (as antigen(s)) one or more tumour antigen as well as other known angiogenic factor(s), for example an angiostatin receptor.

For example, a plasmid encoding the transmembrane extracellular (TMEC) portion of the Her2/neu tumour antigen, a fragment of an oncogene, may act synergistically with a plasmid encoding a human angiomotin molecule so as to reduce tumour development. The TMEC portion may be derived from the transmembrane and extracellular (TMEC) portion of the rat p185 (TMEC), which is the homologue of the human Her2/neu oncogene. This exemplifies how an angiomotin-based vaccine may act in synergy as an “adjuvant” with a tumour antigen, which in this example is derived from Her2/neu but which could be derived from any tumour antigen expressed in human tumours, including but not restricted to examples such as: those derived from the cancer/testis tumour antigens (e.g. the MAGE, BAGE, GAGE, NY-ESO-1 family of antigens); the differentiation antigens (e.g. MART-1/MelanA, MC1R, Gp100, PSA, PSM, Tyrosinase, TRP-1 and -2); the broadly-expressed antigens ART-4, CAMEL, CEA, Cyp-B, hTERT, iCE, MUC1 and 2, PRAME, P15, RUI and 2, SART-1 and 3, WT1); and other more unique or shared antigens (e.g. AFP, b-Catenin, Caspase-8, CDK-4, ELF2, G250, HSP70, HST-2, KIA A0205, MUM-1, 2 and 3, RAGE); viral antigens (e.g. HPV-E7, EBV antigens); or those derived from fusion proteins (e.g. those from bcr-abl, Del-cain, LDL/FUT, TEL/AML1). The tumour antigen may be administered as whole recombinant protein, plasmid, peptide(s), or fragments or parts thereof (e.g. Class I or Class II epitopes).

The vaccine may also further comprise one or more antibody against a tumour antigen or antigenic factor. Hence, the invention also includes angiomotin vaccines combined with antibodies against tumour antigens, including but not restricted to antibodies to the Her2/neu antigen (e.g. Herceptin/Traztusumab) CD20 antigen on lymphomas, EpCAM antigen on colorectal cancer.

The vaccine is intended to generate an immune response against the p130-angiomotin molecule. The resultant immune response can inhibit the formation of new capillaries that is required for the generation and/or sustenance of tumours and other disease states. Further (or alternatively), although p130-angiomotin has not been detected in tumour cells hitherto, small amounts of angiomotin may be expressed by certain malignancies and may therefore serve as a tumour-specific antigen. Whilst not being bound by theory, the present invention encompasses vaccination using a vaccine (i.e. as described herein) where specific T cells and/or antibodies reactive to p130-angiomotin recognize and destroy the tumour cells expressing p130-angiomotin. All modes and approaches for vaccination using angiomotin as a tumour-specific antigen use the same methods as indicated for vaccination for anti-angiogenic effects.

As will be well known to those skilled in the art, the choice of molecule and mode of administration for a vaccine (i.e. an agent acting through the recipient's immune system) differ from those for a direct therapeutic agent. For example, differences between an angiomotin molecule or polynucleotide encoding an angiomotin molecule administered as a vaccine, as opposed to a non-vaccine angiomotin therapeutic entity may include one or more of the following.

A non-vaccine therapeutic entity has to maintain a function of the angiomotin molecule (for example ability to bind angiostatin; or ability to interact with cellular components). The vaccine molecule on the other hand does not have to be a functioning angiomotin molecule, but can be (though does not have to be) the smallest non-functioning derivative (including fragment) or analogue that generates an immune response which is immunologically cross-reactive with the native angiomotin molecule.

Usually a vaccine dose is one or two orders of magnitude lower than a non-vaccine therapeutic dose, for example as measured on a “per kg bodyweight” basis.

Vaccines may include (or be accompanied by) “adjuvants” such as cytokines, BCG and/or alum that boost the immune response. There is no such accompaniment for non-vaccine therapy. An adjuvant may be particularly important in the present case when, for example, immunizing against a “self” protein—i.e. when immunizing against a human protein in a human.

Particularly considering the mechanism of action of the angiomotin molecule (in retarding angiogenesis) non-vaccine therapeutic entities would have to be administered almost daily, as opposed to vaccine administration that involves boosters once in two or three weeks. For example, one or two administrations at intervals of a few weeks may be necessary for either a gene vaccine or a polypeptide/peptide vaccine.

Non-vaccine vectors should have the capability of expressing the functioning molecules (e.g. a polypeptide of the invention) at high levels; this is very difficult to achieve in practice. DNA vaccines can express the immunizing antigen at much lower levels. Many suitable vectors and promoter systems are known, for example a CMV promoter-based system. The immunising antigen can be the minimum non-functional peptide or domain of angiomotin capable and sufficient of generating an immune response (antibody and/or T-cell) against native angiomotin. A functional angiomotin molecule need not be used.

A pharmaceutical composition, medicament or vaccine of the invention may be administered locally, topically, systemically or enterally to generate long-lasting immunity. The resulting immune response hinders the formation of new blood vessels and prevention of neo-vascularisation exerts a prophylactic or therapeutic effect on tumour formation or other vasculogenesis/angiogenesis-related disease.

Fragments of the polypeptide of the invention to be used may comprise at least one domain, i.e. a portion that is capable of, or predicted to be capable of, folding independently in a manner similar to that in which it would fold in the full-length polypeptide. Methods of identifying such domains using computer analysis (for example, incorporating analysis of hydrophobicity and/or likelihood to form an α helix) are well known. The ability to fold independently may be important in generating an antibody response but is not considered important for inducing a T cell response because the polypeptide is degraded and presented as peptide fragments.

One or more peptides or peptidomimetic compounds representing one or more epitope(s) of p130-angiomotin may be used. For example, short peptides, for example of up to about 15, 12, 10 or 9 amino acids (or polynucleotides encoding such short peptides) may be used. By epitopes is included mimotopes, as well known to those skilled in the art.

Epitope sequences may be identified by well known techniques such as those described in Epitope Mapping Protocols (1996) Methods Mol Biol 66 (Glenn E Morris, Ed, Humana Press, Totowa, N.J.; U.S. Pat. No. 4,708,871; Geysen et al., 1984, Proc. Natl. Acad. Sci. USA., 81:3998-4002; Geysen et al., 1986, Mol. Immunol., 23:709-715. Linear or conformational epitopes may be identified using, for example, X-ray crystallography or 2D-nuclear magnetic resonance-derived structural data. Antigenicity or hydrophobicity plots (such as generated using the OMIGA software available from Oxford Molecular Group, based on the algorithms of Hopp et al., 1981, Proc. Natl. Acad. Sci. USA., 78:3824-3828 and Kyte et al., 1982, J. Mol. Biol., 157:105-132) may also be useful in identifying epitopes.

An antibody may be used as an antigen in a vaccine. For example, an antibody to an antibody to the intended target (e.g. angiomotin) is administered and B cells of the immune system make antibodies against that antibody that also recognise the intended target. This is called an anti-idiotype vaccine, and is different from passive antibody therapy, in which an antibody to the intended target is administered.

The invention also provides a combination between a p130-angiomotin-based vaccine and other types of anti-angiogenic therapies targeting endothelial cells or products. Thus, for example, the vaccine may further comprise a polynucleotide or polypeptide (or peptidomimetic compound) suitable for acting as an immunogen against an angiogenesis-promoting polypeptide, for example VEGFR-2 (for example Accession No AF063658) or Tie2 (for example Accession No BC035514). These other angiogenesis-promoting polypeptide-derived sequences (for example VEGFR-2 sequences) may be included in the same or a different polypeptide (or polynucleotide, as appropriate) as the polypeptide/polynucleotide of the invention. The treatment or vaccine may further comprise immunotherapy based on administration of cytokines with anti-tumour effects or of tumour antigens, for example administered as pDNA vaccine, viral vector, or expressed in DC cells or loaded onto DC cells.

The vaccine may comprise further polypeptides or polynucleotides, as will be apparent to those skilled in the art. The polypeptide(s) or polynucleotide(s) may, for example, be included in the vaccine in the form of a recombinant organism or part thereof, or product (such as a cell culture supernatant) thereof, preferably microorganism, preferably capable of expressing the polypeptides(s) i.e. capable of expressing the angiomotin amino acid sequences, or alternatively capable of delivering nucleic acid encoding the polypeptide(s) to a host cell for expression therein. The recombinant microorganism is preferably a non-virulent microorganism, as well known to those skilled in the art. The recombinant microorganism may be, for example, a bifidobacterium or a lactobacillus, or an attenuated Salmonella or BCG or attenuated E. coli. The recombinant organism may alternatively be a plant, for example making use of the teaching of WO97/40177.

In a further alternative, the vaccine can be made either of whole eukaryotic cells or of substances contained by the cells. For example, cells may be derived from the type of organism for which the vaccine is intended. Cells may be recombinant, such as cells derived from a cell line capable of expressing a polypeptide of the invention and/or transfected with a polynucleotide of the invention. Cells may be irradiated, heat-killed or para-formaldehyde-fixed and used for immunization.

The cells may be tumour cells which express p130-angiomotin of the invention, or freshly explanted or cultured endothelial cells, of human origin or xenogeneic from another species, expressing p130-angiomotin or cells transfected with and express p130-angiomotin or parts of this molecule. Alternatively, cells may be antigen presenting cells (APCs), such as dendritic cells (DCs), loaded with the polypeptide of the invention or transfected with the polynucleotide of the invention. A tumour cell vaccine (which may not necessarily comprise a p130-angiomotin antigen) may be used alongside a vaccine of the invention. For a whole cell vaccine, tumour cells are taken out of the patient(s), and grown in the laboratory Then the tumour cells are treated to ensure that 1) they can no longer multiply, and 2) there is nothing present that could infect the patient.

There are two types of whole cell cancer vaccines. An autologous whole cell vaccine is made with the patient's own whole, inactivated tumour cells. An allogenic whole cell vaccine is made with someone else's whole, inactivated tumour cells or several peoples' tumour cells combined.

APC vaccines comprise antigen presenting cells, commonly dendritic cells. Cancer vaccines, for example, can be made of dendritic cells that have been primed, or grown in the presence of, tumour antigens. Dendritic cells (or APCs) primed with antigen carry the tumour antigens on their surface and when injected, may strongly activate T cells.

Antigen vaccines comprise one or more antigens contained in the tumour. Some antigens are common to all cancers of a particular type, and some antigens are unique to an individual. A few antigens are shared between tumours of different types of cancer.

The polypeptide of the invention may be a peptidomimetic compound, for example corresponding to an angiomotin epitope or mimotope. The term “peptidomimetic” refers to a compound that mimics the conformation and desirable features of a particular peptide as a therapeutic agent, but that avoids the undesirable features.

Therapeutic applications involving peptides are limited, due to lack of oral bioavailability and to proteolytic degradation. Typically, for example, peptides are rapidly degraded in vivo by exo- and endo-peptidases, resulting in generally very short biological half-lives. Another deficiency of peptides as potential therapeutic agents is their lack of bioavailability via oral administration. Degradation of the peptides by proteolytic enzymes in the gastrointestinal tract is likely to be an important contributing factor. The problem is, however, more complicated because it has been recognised that even small, cyclic peptides which are not subject to rapid metabolite inactivation nevertheless exhibit poor oral bioavailability. This is likely to be due to poor transport across the intestinal membrane and rapid clearance from the blood by hepatic extraction and subsequent excretion into the intestine. These observations suggest that multiple amide bonds may interfere with oral bioavailability. It is thought that the peptide bonds linking the amino acid residues in the peptide chain may break apart when the peptide drug is orally administered.

There are a number of different approaches to the design and synthesis of peptidomimetics. In one approach, such as disclosed by Sherman and Spatola (1990, J. Am. Chem. Soc., 112:433), one or more amide bond is replaced in an essentially isoteric manner by a variety of chemical functional groups. This stepwise approach has met with some success in that active analogues have been obtained. In some instances, these analogues have been shown to possess longer biological half-lives than their naturally-occurring counterparts. Nevertheless, this approach has limitations. Successful replacement of more than one amide bond has been rare. Consequently, the resulting analogues have remained susceptible to enzymatic inactivation elsewhere in the molecule. When replacing the peptide bond it is preferred that the new linker moiety has substantially the same charge distribution and substantially the same planarity as a peptide bond.

Retro-inverso peptidomimetics (in which the peptide bonds are reversed) can be synthesised by methods known in the art, for example such as those described in Mézière et al., 1997, J. Immunol., 159:3230-3237. This approach involves making pseudopeptides containing changes involving the backbone, and not the orientation of side chains. Retro-inverse peptides, which contain NH—CO bonds instead of CO—NH peptide bonds, are more resistant to proteolysis.

In another approach, a variety of un-coded or modified amino acids such as D-amino acids and N-methyl amino acids have been used to modify mammalian peptides. Alternatively, a presumed bioactive conformation has been stabilised by a covalent modification, such as cyclisation or by incorporation of γ-lactam or other types of bridges. See, e.g. Veber et al., Proc. Natl. Acad. Sci. USA, 75:2636 (1978) and Thursell et al., Biochem. Biophys. Res. Comm., 111:166 (1983).

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

One approach to the synthesis of cyclic stabilised peptidomimetics is ring closing metathesis (RCM). This method involves steps of synthesising a peptide precursor and contacting it with a RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Polypeptides in which one or more of the amino acid residues are chemically modified, before or after the polypeptide is synthesised, may be used as antigen providing that the function of the polypeptide, namely the production of a specific immune response in vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the polypeptide from in vivo metabolism. The polypeptide may be mannosylated or otherwise modified to increase its antigenicity, or combined with a compound for increasing its antigenicity and/or immunogenicity.

The use of agonistic epitopes derived from p130-angiomotin is also included in the present invention. Agonistic epitopes are designed to activate more efficiently the immune system through a more effective activation of MHC class I or MHC class II restricted CD8 or CD4+ T cells. Two general approaches are included in the invention to design agonist epitopes from T cell epitopes. One approach entails modification of HLA anchor residues, resulting in higher HLA class I or class II binding. This approach has been applied with success for several HLA class I binding peptides derived from tumour antigens or microbial antigens. Alternatively, the replacement of residues involved in the T cell receptor (abbreviated TCR) contact may also result in an increased response by T cells and is intended to be covered by this invention.

Generally, as well known to those skilled in the art, and as described in, for example, U.S. Pat. No. 5,869,445, amino acid substitutions may be made in a variety of ways to provide other embodiments of variants within the present invention. First, for example, amino acid substitutions may be made conservatively; i.e., a substitute amino acid replaces an amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. An example of a non-conservative change is to replace an amino acid of one group with an amino acid from another group.

Another way to make amino acid substitutions to produce variants of the present invention is to identify and replace amino acids in T cell motifs with potential to bind to class II MHC molecules (for CD4+ T cell response) or class I MHC molecules (for CD8+ T cell response). Peptide segments with a motif with theoretical potential to bind to class II MHC molecules may be identified by computer analysis. For example, a protein sequence analysis package, T Sites, that incorporates several computer algorithms designed to distinguish potential sites for T cell recognition can be used (Feller and de la Cruz, 1991, Nature 349:720-721). Two searching algorithms are used: (1) the AMPHI algorithm described in Feller and de la Cruz, 1991, Nature 349:720-721; Margalit et al., 1987, J. Immunol. 138:2213-2229, identifies epitope motifs according to alpha-helical periodicity and amphipathicity; (2) the Rothbard and Taylor algorithm identifies epitope motifs according to charge and polarity pattern (Rothbard and Taylor, 1988, EMBO J., 7:93-100). Segments with both motifs are most appropriate for binding to class II MHC molecules. CD8+ T cells recognize peptide bound to class I MHC molecules. Falk et al. have determined that peptides binding to particular MHC molecules share discernible sequence motifs (Falk et al., 1991, Nature, 351:290-296). A peptide motif for binding in the groove of HLA-A2.1 has been defined by Edman degradation of peptides stripped from HLA-A2.1 molecules of a cultured cell line (Table 2, from Falk et al., supra). The method identified the typical or average HLA-A2.1 binding peptide as being 9 amino acids in length with dominant anchor residues occurring at positions 2 (L) and 9 (V). Commonly occurring strong binding residues have been identified at positions 2 (M), 4 (E,K), 6 (V), and 8 (K). The identified motif represents the average of many binding peptides.

The epitope(s) (for example epitope-forming amino acid sequences, or regions considered to comprise anti-angiogenic epitopes) may be present as single copies or as multiples, for example tandem repeats. Such tandem or multiple repeats may be sufficiently antigenic themselves to obviate the use of a carrier. It may be advantageous for the polypeptide to be formed as a loop, with the N-terminal and C-terminal ends joined together, or to add one or more Cys residues to an end to increase antigenicity and/or to allow disulphide bonds to be formed. If the epitope, for example epitope-forming amino acid sequence, is covalently linked to a carrier, preferably a polypeptide, then the arrangement is preferably such that the epitope-forming amino acid sequence forms a loop.

According to current immunological theories, a carrier function should be present in any immunogenic formulation in order to stimulate, or enhance stimulation of, the immune system. The epitope(s) as defined above in relation to the preceding aspects of the invention may be associated, for example by cross-linking, with a separate carrier, such as serum albumins, myoglobins, bacterial toxoids and keyhole limpet haemocyanin. The polypeptide of the invention may itself act as a carrier or adjuvant. More recently developed carriers which induce T-cell help in the immune response include the hepatitis-B core antigen (also called the nucleocapsid protein), is presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both.

Alternatively, several copies of the same or different epitope may be cross-linked to one another; in this situation there is no separate carrier as such, but a carrier function may be provided by such cross-linking. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present). Any of the conventional ways of cross-linking polypeptides may be used, such as those generally described in O'Sullivan et al., 1979, Anal. Biochem., 100:100-108. For example, the first portion may be enriched with thiol groups and the second portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP), a heterobifunctional cross-linking agent which incorporates a disulphide bridge between the conjugated species. Amide and thioether bonds, for example achieved with m-maleimidobenzoyl-N-hydroxysuccinimide ester, are generally more stable in vivo than disulphide bonds.

Further useful cross-linking agents include S-acetylthioglycolic acid N-hydroxysuccinimide ester (SATA) which is a thiolating reagent for primary amines which allows deprotection of the sulphydryl group under mild conditions (Julian et al., 1983, Anal. Biochem., 132:68), dimethylsuberimidate dihydrochloride and N,N′-o-phenylenedimaleimide.

If the polypeptide is prepared by expression of a suitable nucleotide sequence in a suitable host, then it may be advantageous to express the polypeptide as a fusion product with a peptide sequence which acts as a carrier. Kabigen's “Ecosec” system is an example of such an arrangement.

Other adjuvants that may be useful include adjuvants discussed in WO 02/053181, for example VSA3, which includes DDA (see U.S. Pat. No. 5,951,988).

Suitable vectors or constructs which may be used to prepare a suitable recombinant polypeptide or polynucleotide will be known to those skilled in the art. A polynucleotide capable of expressing the required polypeptide or polypeptides may be prepared using techniques well known to those skilled in the art.

It may be desirable for the polynucleotide to be capable of expressing the polypeptide(s) in the recipient, so that the human or animal may be administered the polynucleotide, leading to expression of the antigenic polypeptides (i.e. sequences derived from angiomotin and optionally other polypeptides) in the human or animal. The polypeptide(s), for example p130-angiomotin, may be expressed from any suitable polynucleotide (genetic construct) as is described below and delivered to the recipient. Typically, the genetic construct which expresses the polypeptide comprises a polynucleotide sequence encoding said polypeptide, operatively linked to a promoter which can express the transcribed polynucleotide (e.g. mRNA) molecule in a cell of the recipient, which may be translated to synthesise the said polypeptide. Suitable promoters will be known to those skilled in the art, and may include promoters for ubiquitously expressed genes, for example housekeeping genes or for tissue-selective genes, depending upon where it is desired to express the said polypeptide (for example, in dendritic cells or other antigen presenting cells or precursors thereof). Preferably, a dendritic cell or dendritic precursor cell-selective promoter is used, but this is not essential, particularly if delivery or uptake of the polynucleotide is targeted to the selected cells, e.g. dendritic cells or precursors, Dendritic cell-selective promoters may include the CD83 or CD36 promoters.

Other polypeptides/proteins which may desirably be expressed or combined with a vaccine of the invention include immunostimulatory agents such as cytokines or growth factors. Examples include GM-CSF, IL-2, IL12 or IL-15. Other immunostimulatory agents which may be expressed or included are factors that bind to so called Toll receptors, which include the small immunostimulatory molecule Imiquimod, microbial products such as flagellin or unmethylated bacterial CpG motifs or oligonucleotides based on CpG motifs.

The polynucleotide sequence capable of expressing the polypeptide(s) is preferably operatively linked to regulatory elements necessary for expression of said sequence.

“Operatively linked” refers to juxtaposition such that the normal function of the components can be performed. Thus, a coding sequence “operatively linked” to regulatory elements refers to a configuration wherein the nucleic acid sequence encoding the antigen (or immunostimulatory molecule) can be expressed under the control of the regulatory sequences.

“Regulatory sequences” refers to nucleic acid sequences necessary for the expression of an operatively linked coding sequence in a particular host organism. For example, the regulatory sequences which are suitable for eukaryotic cells are promoters, polyadenylation signals, and enhancers.

“Vectors” means a DNA molecule comprising a single strand, double strand, circular or supercoiled DNA. Suitable vectors include retroviruses, adenoviruses, adeno-associated viruses, pox viruses and bacterial plasmids. Retroviral vectors are retroviruses that replicate by randomly integrating their genome into that of the host. Suitable retroviral vectors are described in WO 92/07573.

Viral vectors are intended not to make people sick or to carry any diseases. These viruses can be engineered in the laboratory so that when they infect a human cell, the cell will make and display the required antigen on its surface. The virus is capable of infecting only a small number of human cells—enough to start an immune response, but not enough to make a person sick.

Viruses can also be engineered to make cytokines or display proteins on their surface that help activate immune cells. These can be given alone or with a vaccine to help the immune response.

Adenovirus comprises a linear double-stranded DNA genome. Suitable adenoviral vectors are described in Rosenfeld et al., 1991, Science, 252:432.

Adeno-associated viruses (AAV) belong to the parvo-virus family and comprise a single-stranded DNA genome of about 4-6 KB.

Pox viral vectors are large viruses and have several sites in which genes can be inserted. They are thermo-stable and can be stored at room temperature. Safety studies indicate that pox viral vectors are replication-defective and cannot be transmitted from host to host or to the environment.

Targeting the vaccine to specific cell populations, for example APCs, may be achieved, for example, either by the site of injection, use of targeting vectors and delivery systems, or selective purification of such a cell population from the recipient and ex vivo administration of the peptide or nucleic acid (for example dendritic cells may be sorted as described in Zhou et al., 1995, Blood, 86:3295-3301; Roth et al., 1996, Scand. J. Immunology 43, 646-651. In addition, targeting vectors may comprise a tissue- or tumour-selective promoter which directs expression of the antigen at a suitable place.

Although the genetic construct comprising the polynucleotide of the invention can be DNA or RNA it is preferred if it is DNA. Preferably, the genetic construct is adapted for delivery to a human cell.

Means and methods of introducing a genetic construct into a cell in or removed from an animal body are known in the art. For example, the constructs of the invention may be introduced into the cells by any convenient method, for example methods involving retroviruses, so that the construct is inserted into the genome of the (dividing) cell. Targeted retroviruses are available for use in the invention; for example, sequences conferring specific binding affinities may be engineered into pre-existing viral env genes (see Miller & Vile, 1995, Faseb J., 9:190-199 for a review of this and other targeted vectors for gene therapy).

Preferred retroviral vectors may be lentiviral vectors such as those described in Verma a & Somia, 1997, Nature, 389:239-242.

Other methods involve simple delivery of the construct into the cell for expression therein either for a limited time or, following integration into the genome, for a longer time. An example of the latter approach includes liposomes (Nassander et al., 1992, Cancer Res., 52:646-653). Other methods of delivery include adenoviruses carrying external DNA via an antibody-polylysine bridge (see Curiel Prog. Med. Virol., 40:1-18) and transferrin-polycation conjugates as carriers (Wagner et al., 1990, Proc. Natl. Acad. Sci. USA, 87:3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

Bacterial delivery methods which may be suitable are described in Dietrich, 2000, Antisense Nucleic Acid Drug Delivery, 10:391-399. For example, attenuated bacterial strains allow the administration of recombinant vaccines via the mucosal surfaces. Whereas attenuated bacteria are generally engineered to express heterologous antigens, a further approach employs intracellular bacteria for the delivery of eukaryotic antigen expression vectors (DNA vaccines). This strategy allows a direct delivery of DNA to professional antigen-presenting cells (APC), such as macrophages and dendritic cells (DC), through bacterial infection. The bacteria used for DNA vaccine delivery either enter the host cell cytosol after phagocytosis by the APC, for example, Shigella and Listeria, or they remain in the phagosomal compartment, such as Salmonella. Both intracellular localizations of the bacterial carriers may be suitable for successful delivery of DNA vaccine vectors of the present invention.

Expression of the angiomotin polypeptide of the invention may be under the control of inducible bacterial promoters, for example promoters that are induced when the bacterium encounters or enters a host organism environment (for example the host's gut) or binds to or enters a host cell.

The polynucleotide of the invention can be administered as a “gene-gun” intradermal vaccination, intramuscular injection, or plasmid DNA vaccination injected intradermally or intramuscularly and then followed by cutaneous or intramuscular “electroporation” at the site of injection, to increase the efficacy of the vaccination.

The DNA may also be delivered by adenovirus wherein it is present within the adenovirus particle, for example, as described below.

A high-efficiency nucleic acid delivery system that uses receptor-mediated endocytosis to carry DNA macromolecules into cells may be employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the target cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al., 1992, Proc. Natl. Acad. Sci. USA, 89:6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

“Naked DNA” and DNA complexed with cationic and neutral lipids may also be useful in introducing the DNA of the invention into cells of the recipient. Non-viral approaches to gene therapy are described in Ledley, 1995, Human Gene Therapy, 6:1129-1144. Alternative targeted delivery systems are also known such as the modified adenovirus system described in WO 94/10323 wherein, typically, the DNA is carried within the adenovirus, or adenovirus-like, particle. Michael et al., 1995, Gene Therapy, 2:660-668 describes modification of adenovirus to add a cell-selective moiety into a fibre protein. Mutant adenoviruses which replicate selectively in p53-deficient human tumour cells, such as those described in Bischoff et al., 1996, Science, 274:373-376 are also useful for delivering the genetic construct of the invention to a cell. Other suitable viruses or virus-like particles include HSV, AAV, vaccinia, lentivirus and parvovirus.

Immunoliposomes (antibody-directed liposomes) are especially useful in targeting to cell types which over-express a cell surface protein for which antibodies are available, as is possible with dendritic cells or precursors, for example using antibodies to CD1, CD14 or CD83 (or other dendritic cell or precursor cell surface molecule, as indicated above). For the preparation of immuno-liposomes MPB-PE (N-[4-(p-maleimidophenyl)butyryl]-phosphatidylethanolamine) is synthesised according to the method of Martin & Papahadjopoulos, 1982, J. Biol. Chem., 257:286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from un-conjugated antibodies by ultracentrifugation at 80 000×g for 45 min. Immunoliposomes may be injected, for example intraperitoneally or directly into a site where the target cells are present, for example subcutaneously.

It will be appreciated that it may be desirable to be able to regulate temporally expression of the polypeptide(s) (for example antigenic polypeptides) in the cell. Thus, it may be desirable that expression of the polypeptide(s) is directly or indirectly (see below) under the control of a promoter that may be regulated, for example by the concentration of a small molecule that may be administered to the recipient when it is desired to activate or repress (depending upon whether the small molecule effects activation or repression of the said promoter) expression of the polypeptide. It will be appreciated that this may be of particular benefit if the expression construct is stable i.e. capable of expressing the polypeptide (in the presence of any necessary regulatory molecules) in the said cell for a period of at least one week, one, two, three, four, five, six, eight months or one or more years. It is preferred that the expression construct is capable of expressing the polypeptide in the said cell for a period of less than one month. A preferred construct of the invention may comprise a regulatable promoter. Examples of regulatable promoters include those referred to in the following papers: Rivera et al., 1999, Proc. Natl. Acad. Sci. USA, 96:8657-62 (control by rapamycin, an orally bioavailable drug, using two separate adenovirus or adeno-associated virus (AAV) vectors, one encoding an inducible human growth hormone (hGH) target gene, and the other a bipartite rapamycin-regulated transcription factor); Magari et al., 1997, J. Clin. Invest., 100:2865-72 (control by rapamycin); Bueler, 1999, Biol. Chem., 380:613-22 (review of adeno-associated viral vectors); Bohl et al., 1998, Blood, 92:1512-7 (control by doxycycline in adeno-associated vector); Abruzzese et al., 1996, J. Mol. Med., 74:379-92 (reviews induction factors e.g., hormones, growth factors, cytokines, cytostatics, irradiation, heat shock and associated responsive elements). Tetracycline—inducible vectors may also be used. These are activated by a relatively-non toxic antibiotic that has been shown to be useful for regulating expression in mammalian cell cultures. Also, steroid-based inducers may be useful especially since the steroid receptor complex enters the nucleus where the DNA vector must be segregated prior to transcription.

This system may be further improved by regulating the expression at two levels, for example by using a tissue-selective promoter and a promoter controlled by an exogenous inducer/repressor, for example a small molecule inducer, as discussed above and known to those skilled in the art. Thus, one level of regulation may involve linking the appropriate polypeptide-encoding gene to an inducible promoter whilst a further level of regulation entails using a tissue-selective promoter to drive the gene encoding the requisite inducible transcription factor (which controls expression of the polypeptide (for example the antigenic polypeptide)-encoding gene from the inducible promoter). Control may further be improved by cell-type-specific targeting of the genetic construct.

The genetic constructs of the invention can be prepared using methods well known in the art.

The therapeutic agent or molecule (vaccine), for example antigenic molecule, for example a p130-angiomotin polypeptide and/or its encoding polynucleotide and/or an antibody of the invention or a formulation thereof, may be administered by any conventional method including oral and parenteral (eg subcutaneous or intramuscular) injection. Preferred routes include oral, intranasal or intramuscular injection. The treatment may consist of a single dose or a plurality of doses over a period of time. It will be appreciated that an inducer, for example small molecule inducer as discussed above may preferably be administered orally.

Methods of delivering genetic constructs, for example adenoviral vector constructs comprising an encoding polynucleotide (or an antisense polynucleotide) of the invention to cells of a recipient will be well known to those skilled in the art. In particular, an adoptive therapy protocol may be used or, more preferably, a gene gun may be used to deliver the construct to dendritic cells, for example in the skin.

Adoptive therapy protocols are described in Nestle et al., 1998, Nature Med., 4:328-332 and De Bruijn et al., 1998, Cancer Res., 58:724-731.

The therapeutic agent (vaccine) may be given to a subject who is being treated for the disease by some other method. Thus, although the method of treatment may be used alone it is desirable to use it as an adjuvant therapy, for example alongside conventional preventative or therapeutic methods or immunotherapy targeting tumour antigens. For example, combinations of the vaccine of the invention with vaccines based on tumour antigens, administered as peptides, proteins, oligonucleotides or whole tumour cells may be suitable combination therapies.

Whilst it is possible for a therapeutic molecule as described herein, for example an antigenic molecule or immunostimulatory molecule, to be administered alone, it is preferable to present it as a pharmaceutical formulation, together with one or more acceptable carriers. The carrier(s) must be “acceptable” in the sense of being compatible with the therapeutic molecule (which may be a nucleic acid or polypeptide) and not deleterious to the recipients thereof. Typically, the carriers will be water or saline which will be sterile and pyrogen free.

The pharmaceutical composition may further comprise a component for increasing the antigenicity and/or immungenicity of the composition, for example an adjuvant and/or a cytokine, as discussed above. A polyvalent antigen (cluster of antigens) may be useful.

Commercial versions of cytokines have been used both as nonspecific immunotherapies to generally boost the immune system and as adjuvants given along with other immunotherapies such as tumor vaccines. GM-CSF is being tested against cancer as a nonspecific immunotherapy and as an adjuvant given with other types of immunotherapies.

A variety of other compounds are known to boost the activity of the immune system and are now under study as possible adjuvants, particularly for vaccine therapies. Some of the most commonly studied adjuvants are listed below, but many more are under development.

Levamisole, a drug first used against parasitic infections, has recently been found to improve survival rates among people with colorectal cancer when used together with some chemotherapy drugs. It is often used as an immunotherapy adjuvant because it can activate T lymphocytes. Levamisole is now used routinely for people with some stages of colorectal cancer and is being tested in clinical trials as a treatment for other types of cancer.

Aluminum hydroxide (alum) is one of the most common adjuvants used in clinical trials for cancer vaccines. It is already used in vaccines against several infectious agents, including the hepatitis B virus.

Bacille Calmette-Guérin (BCG) is a bacterium that is related to the bacterium that causes tuberculosis. The effect of BCG infection on the immune system makes this bacterium useful as a form of anticancer immunotherapy. BCG was one of the earliest immunotherapies used against cancer. It is FDA-approved as a routine treatment for superficial bladder cancer. Its usefulness in other cancers as a nonspecific adjuvant is also being tested. Researchers are looking at injecting BCG to give an added boost to the immune system when using chemotherapy, radiation therapy, or other types of immunotherapy.

Incomplete Freund's Adjuvant (IFA) is given together with some experimental therapies to help stimulate the immune system and to increase the immune response to cancer vaccines. IFA is a liquid consisting of an emulsifier in white mineral oil.

QS-21 is a relatively new immune stimulant made from a plant extract that increases the immune response to vaccines used against melanoma.

DETOX is another relatively new adjuvant. It is made from parts of the cell walls of bacteria and a kind of fat. It is used with various immuno-therapies to stimulate the immune system.

Keyhole limpet hemocyanin (KLH) is another adjuvant used to boost the effectiveness of cancer vaccine therapies. It is extracted from a type of sea mollusc.

Dinitrophenyl (DNP) is a hapten/small molecule that can attach to tumour antigens and cause an enhanced immune response. It is used to modify tumour cells in certain cancer vaccines.

An eighth aspect of the invention provides a use of an antibody or fragment of the invention in the detection and/or measurement of angiogenesis and/or tumour formation in a test sample. For example, test samples such as serum, tumour protein extracts or mRNA extracted from tumours may be used to quantify levels of p80- and/or p130-angiomotin polynucleotide and/or polypeptide and/or antibodies against p80- and/or p130-angiomotin. Many well-known methods could be used for this purpose, including ELISA and/or real-time polymerase chain reaction (PCR) assays. It will be understood that the detection and/or measurement of angiogenesis and/or tumour formation may be performed using in vitro and/or in vivo methods dependent on the test sample to be analysed.

A ninth aspect of the invention provides a pharmaceutical composition for modulating angiogenesis and/or tumour formation, comprising an effective amount of an angiomotin polypeptide of the invention and/or its encoding polynucleotide (and/or antisense polynucleotide) and/or an antibody or fragment of the invention, and a pharmaceutical excipient or diluent.

By “effective amount” we include an amount that is sufficient to modulate angiogenesis and/or tumour formation. Methods for measuring the level of angiogenesis and/or tumour formation in a subject, such as a human, are well known to those skilled in the art.

For example, tumour formation can be evaluated in murine tumour cell lines such as the mouse mammary carcinoma line D2F2, and lymphoma line, EL-4. Tumour formation in vivo can be determined in model systems such as the mouse transgene breast cancer model, BALB-neuT which spontaneously develop tumours as a result of over-expressing the transforming rat Her.2/neu oncogene under control of the mouse mammary tumour virus promoter (Boggio et al., 1998, J. Exp. Med., 188:589-96).

The status of the host immune system in these models may be assessed by using known immunological assays measuring CD8+ and CD4+ T cell responses, such as ELISPOT assays, cytokine release assays and T cell proliferation assays as well as assays to measure antibody responses, such as ELISA assays and flow cytometry-based assays. Neo-vascularisation in the tumour may be assessed by various known assays, including the “Matrigel-plug” assay (as described in, for example, London et al., 2003, Cancer Gene Ther., 10:823-832) or imaging-based assays such as the skin-flap window-chamber model.

An effective amount may be determined in vivo by use of such methods to determine the level of angiogenesis in the subject before and after treatment with an amount of the polypeptide and/or its encoding polynucleotide (and/or antisense polynucleotide) and/or an antibody or fragment of the invention. Alternatively, an idea of the effective range of a medicament may be obtained by testing the medicament in vitro using, for example, the methods described in the Examples.

Preferably, the pharmaceutical composition prevents and/or reduces angiogenesis and/or tumour formation.

A tenth aspect of the invention provides a vaccine for modulating angiogenesis and/or tumour formation, comprising an effective amount of an angiomotin polypeptide of the invention and/or its encoding polynucleotide (and/or antisense polynucleotide), and an excipient or diluent.

There are a number of animal studies which demonstrate that a vaccine candidate may serve both prophylactically (i.e. to prevent tumours in an animal challenged with tumour cells) and therapeutically (i.e. administration of the vaccine causes regression of previously established tumours), for example: Cavallo et al., 1993, Cancer Res., 21:5067; Nanni et al., 2001, J. Exp. Med., 194:1195.

Preferably, the vaccine of the invention or pharmaceutical composition of the invention further comprises at least one additive for assisting or augmenting the action of the polypeptide and/or polynucleotide (and/or antisense polynucleotide) and/or antibody or fragment therein.

More preferably, the at least one additive is an immunostimulatory molecule, conveniently, a cytokine or polynucleotide (and/or antisense polynucleotide) encoding a cytokine.

Preferably, the vaccine of the invention or pharmaceutical composition of the invention comprises a cell or cell extract which is, more preferably, an APC which is loaded with the angiomotin polypeptide of the invention or transfected with its encoding polynucleotide (and/or antisense polynucleotide) of the invention.

Conveniently the cell is a tumour cell expressing angiomotin or an endothelial cell expressing angiomotin.

An eleventh aspect of the invention provides a method of generating an immune response against angiomotin polypeptide of the invention in a mammal, the method comprising the steps of:
(i) stimulating ex vivo immune cells collected from the mammal with an angiomotin polypeptide of the invention or its encoding polynucleotide (and/or antisense polynucleotide);
(ii) transferring the stimulated immune cells back into the mammal, such that transfer of the cells back into the mammal generates and immune response against angiomotin.

Preferably, the mammal is a human.

Preferably, the immune response serves prophylactically or therapeutically to inhibit the onset or progress of an angiogenesis-related disease.

In this aspect of the invention, a subject's immune cells are stimulated ex vivo (i.e. outside the subject's body) by a polypeptide of the invention. This polypeptide may be presented to the patient's immune cells, in particular T cells, following transfection of a polynucleotide of the invention, into antigen presenting cells (APCs). The antigen presenting cell could also be pre-treated externally with the polypeptide of the invention or peptides derived from it. Any cell type with the capacity to stimulate lymphocytes are here operationally defined as antigen presenting cell; these are considered to include dendritic cells (DCs) derived from monocytes or lymphoid cells of the bone-marrow; and B cells, stimulated with mitogens or immortalized by, for example, Epstein Barr Virus (EBV).

Adoptive transfer of the stimulated immune cells back into the patient may result in the inhibition of neo-angiogenesis through recognition of angiomotin expressed in the endothelial cells by the transferred immune cells, mainly T cells of CD8+ or CD4+ type and consequently restriction of the progress of tumours or other angiogenesis-related disease. Adoptive transfer of the stimulated immune cells back into the patient may also result in the restriction of the progress of tumours through recognition of angiomotin expressed in the tumour cells as a tumour antigen by the transferred immune cells, mainly T cells of CD8+ or CD4+ type.

Preferably, in the uses, pharmaceutical compositions, vaccines or methods of the invention, the encoding polynucleotide comprises:
(i) the polynucleotide (and/or antisense polynucleotide) of SEQ ID NO:2; or
(ii) a polynucleotide (and/or antisense polynucleotide) which has at least 80% and/or at least 90% and/or at least 95% and/or at least 98% identity to SEQ ID NO:2 and/or is capable of hybridising to SEQ ID NO:2 under conditions of 2×SSC at 65° C. and/or which encodes a functional polypeptide; or
(iii) a fragment of SEQ ID NO:2 which encodes a functional polypeptide fragment.
   and wherein the polynucleotide (and/or antisense polynucleotide) does not encode a p80-angiomotin or a fragment of p80-angiomotin.

Polynucleotides and/or antisense polynucleotides of the invention do not include those identical to polynucleotides encoding p80-angiomotin polypeptides and fragments thereof.

The polynucleotide of the invention may further comprise regulatory sequences and/or vector sequences as described above to permit its replication and/or expression in the necessary host and/or target cell.

It is well known in the art that two nucleic acids encoding the same gene may have similar but non-identical nucleotide sequences. A variation in the nucleotide sequence of a gene is one which is (i) usable to produce a protein or a fragment thereof which is in turn usable to prepare antibodies which specifically bind to the protein encoded by the said gene or (ii) an antisense sequence corresponding to the gene or to a variation of type (i) as just defined. For example, different codons can be substituted which code for the same amino acid(s) as the original codons. Alternatively, the substitute codons may code for a different amino acid that will not affect the activity or immunogenicity of the protein or which may improve its activity or immunogenicity. For example, site-directed mutagenesis or other techniques can be employed to create single or multiple mutations, such as replacements, insertions, deletions, and transpositions, as described in Botstein et al., 1985, Science, 229:193-1210, which is incorporated herein by reference. Since such modified genes can be obtained by the application of known techniques to the teachings contained herein, such modified genes are within the scope of the claimed invention.

Moreover, it will be recognised by those skilled in the art that the gene sequence (or fragments thereof) of the invention can be used to obtain other DNA sequences that hybridise with it under conditions of high stringency. Such DNA includes any genomic DNA. Accordingly, the gene of the invention includes DNA that encodes an amino acid sequence with more than 50% identity to the deduced amino acid sequence of the gene identified in the method of the invention, or a DNA sequence that shows at least 55 percent, preferably 60 percent, and most preferably 70 percent homology with the gene identified in the method of the invention, provided that such homologous DNA is usable in the methods of the present invention. The gene of the invention also includes DNA that encodes an amino acid sequence with more than 20% identity to a sequence of at least 200 amino acids of bovine rod opsin.

DNA-DNA, DNA-RNA and RNA-RNA hybridisation may be performed in aqueous solution containing between 0.1×SSC and 6×SSC and at temperatures of between 55° C. and 70° C. It is well known in the art that the higher the temperature or the lower the SSC concentration the more stringent the hybridisation conditions. By “high stringency” we mean 2×SSC and 65° C. 1×SSC is 0.15M NaCl/0.015M sodium citrate.

Variations of the gene include genes in which relatively short stretches (for example 20 to 50 nucleotides) have a high degree of homology (at least 50% and preferably at least 90 or 95%) with equivalent stretches of the gene of the invention even though the overall homology between the two genes may be much less. This is because important active or binding sites may be shared even when the general architecture of the protein is different.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperatures. The higher the degree of desired homology between the probe and hybridisable sequence, the higher the relative temperature that can be used. For additional details and explanation of stringency of hybridization reactions, see Ausubel et al., 1995, Current Protocols in Molecular Biology (Wiley Interscience Publishers) or Protocols Online URL: www.protocol-online.net/molbio/index.htm).

“Stringent conditions” or high-stringency may be identified by those that: (1) use low ionic strength and high temperature for washing, for example 0.1×SSC, 0.2% SDS @ 65-70° C.

“Moderately-stringent conditions” may be identified as described by Sambrook et al., 2001, Molecular Cloning: A Laboratory Manual (3rd Edition) and include the use of washing solution and hybridization conditions (e.g. temperature, ionic strength, and % SDS) less stringent that those described above. An example of moderately stringent conditions is 0.2×SSC, 0.1% SDS @ 58-65° C. The skilled artisan will recognize how to adjust temperature, ionic strength, etc. as necessary to accommodate factors such as probe length, degree of homology between probe and target site and the like. Therefore, in addition to the sequence of interest, it is contemplated that additional or alternative probe sequences which vary from that of the sequence of interest will also be useful in screening for the sequence of interest.

Preferred, non-limiting examples which embody certain aspects of the invention will now be described, with reference to the following figures:

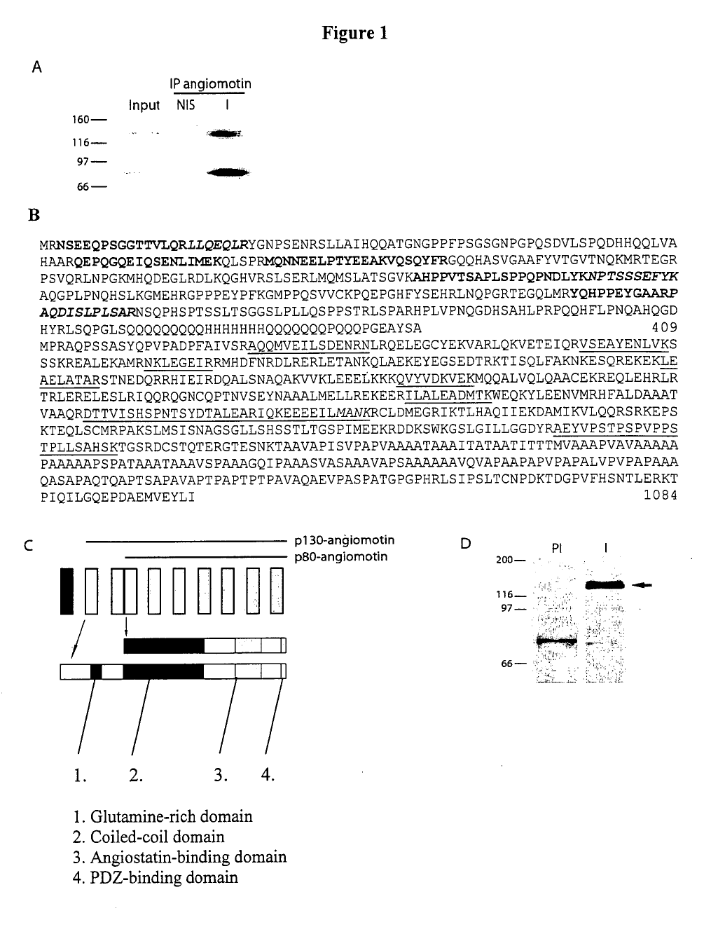

FIG. 1: Identification of p130-angiomotin, a novel splice isoform of angiomotin. A: Rabbit polyclonal antibodies directed at the 24aa C-terminus of angiomotin recognise two proteins of 80 kDa and 130 kDa in 293T cells. NIS=non immune serum. B: 293T cells were subjected to immunoprecipitation, separated on SDS PAGE and proteins visualised by silver staining. The 80 kDa and the 130 kDa bands were extracted and analysed by mass spectrometry. Red (bold text) represents sequences from proteolytic fragments that are unique to the p130 isoform. Sequences in blue (underlined text) (410-1084) correspond to peptides that were also found in the p80 protein. Italics were used when concurrent sequences were identified to distinguish individual peptide fragments. C: a new open reading frame is generated by alternative splicing between exons 2 and 3 which results in the N-terminal extension of p130-angiomotin. Blue colour (1) corresponds to the glutamine-rich domain in p130-angiomotin, red (2) colour to the coiled-coil, yellow (3) to the angiostatin binding domain and turquoise colour (4) to the PDZ binding domain. D: rabbit polyclonal antibodies generated against 266 amino acids in the N-terminus of p130-angiomotin specifically detect the 130 kDa isoform of angiomotin as analysed by western blot. PI=preimmune sera, I=immune sera.

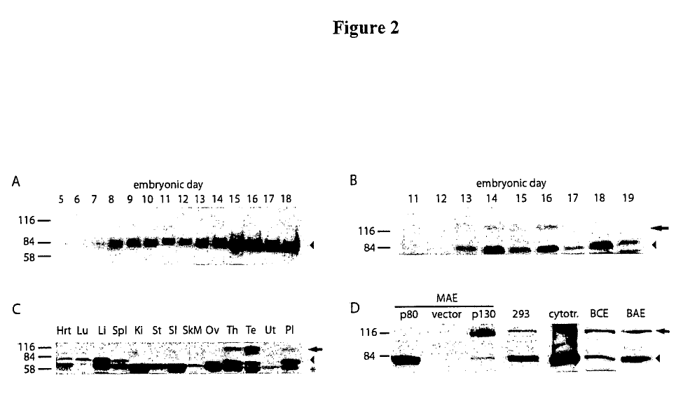

FIG. 2: Expression of p130-angiomotin in embryonic and adult mouse tissues. A: Western blot analysis of angiomotin protein levels in mouse embryos from embryonic day 5 to 19. p80-angiomotin was expressed from embryonic day 5 to 18 (arrowhead) whereas no p130-angiomotin protein could be detected. B: Western blot analysis of mouse placental lysates show expression of p80-angiomotin from embryonic day 11 to 19 (arrowhead). P130-angiomotinis expressed from embryonic day 13 to 16 (arrow). C: Analysis of mouse adult tissues show expression of p80-angiomotin in heart, lung, liver, spleen, ovary, liver, thymus, testis and placenta (arrowhead). P130-angiomotin is only found in thymus, testis and placenta (arrowhead). The asterisk indicates non-specific bands. Lu, lung; Li, liver; Spl, spleen, Ki, kidney; St, stomach; SI, small intestine; SkM, skeletal muscle; Ov, ovary; Th, thymus; Te, testis; Ut, uterus, Pl, placenta. D: Western blot analysis of MAE cells retrofected with vector, p-80 angiomotin and p-130 angiomotin and endogenous expression of both isoforms in 293T cells, cytotrophoblasts, hTERT⁺-BCE cells and BAE cells. The arrows indicate the location of p130 and p80 bands.

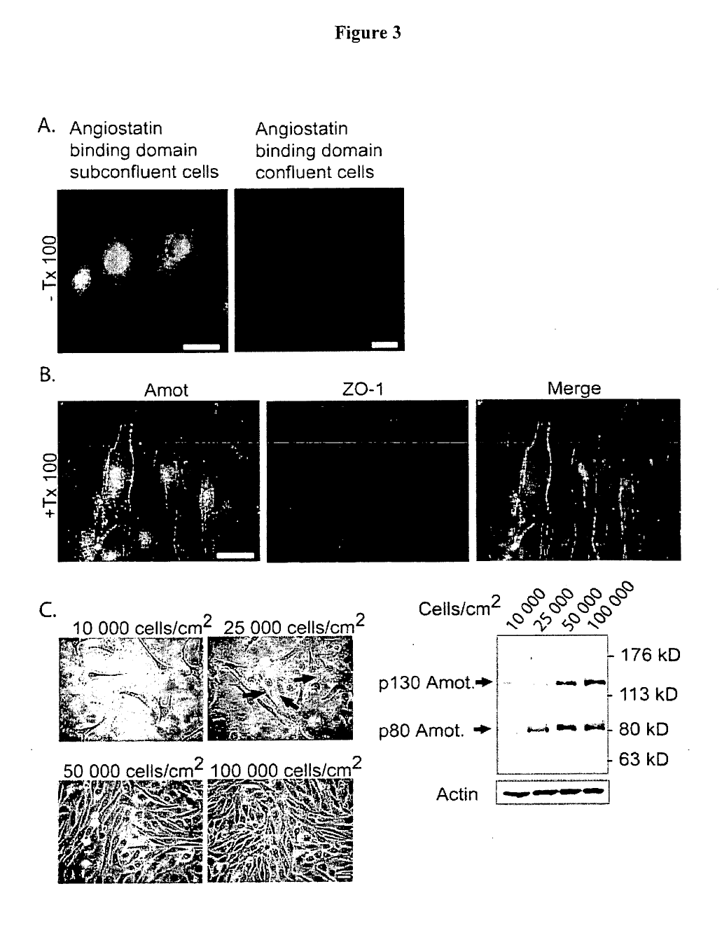

FIG. 3: Angiomotin co-localises with ZO-1 in primary bovine capillary endothelial cells. A: The antibody against the angiostatin binding domain (green) binds to angiomotin on the cell surface of sub-confluent bovine capillary endothelial (BCE) cells. An antibody against an intracellular epitope of caveolin (red) was used as a control for an intact cell membrane. Cell surface staining was lost when cells were confluent. B: Endogenous angiomotin co-localises with ZO-1 in confluent BCE cells. Confluent BCE cells were permeabilised with Triton X-100 and stained with the antibody against the C-terminus of angiomotin (green) and antibody against ZO-1 (red). Overlay of the two images is shown (merge) with co-localisation appearing as yellow. C: Expression of p80 and p130 isoforms of angiomotin is regulated by cell density. Cells were plates at the indicated densities and analysed for angiomotin expression by Western blot. The bar represents 20 μm.

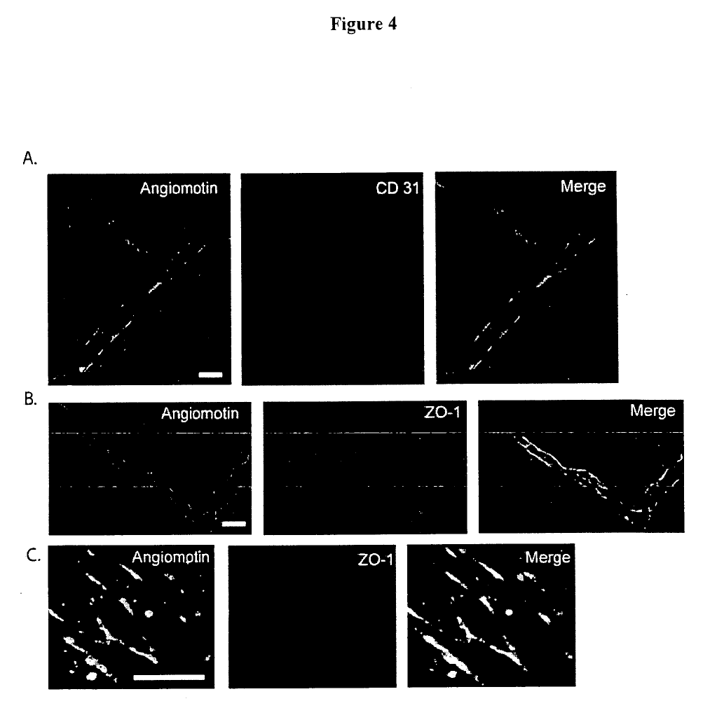

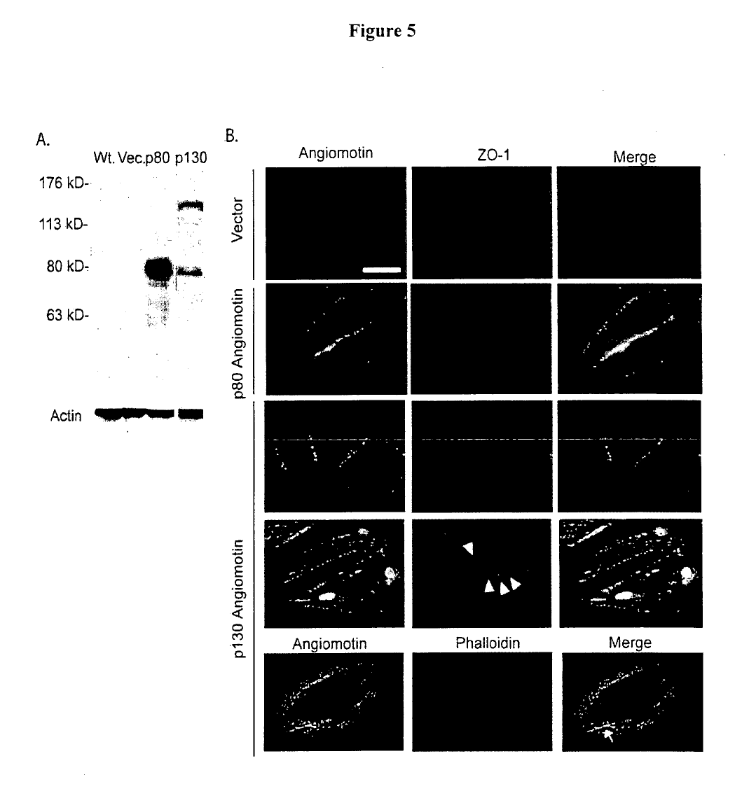

FIG. 4: Angiomotin co-localises with ZO-1 in blood vessels during vascularisation of the postnatal mouse retina. Mouse retinas from postnatal day 5 were analysed by whole-mount fluorescence microscopy using antibodies against ZO-1 (red), CD31/PECAM (red) and antibody against the C-terminus of angiomotin (green). Overlay of the two images is shown (merge) with co-localisation appearing as yellow. A: Angiomotin is expressed in blood vessels as indicated by the endothelial marker CD31/PECAM. B: Angiomotin co-localises with ZO-1 in cell-cell contacts in retinal blood vessels. C: Enlargement of a part of a vessel showing co-localisation of angiomotin and ZO-1 in cell-cell contacts. The bar represents 10 μm.

FIG. 5: Angiomotin co-localises with ZO-1 in Chinese Hamster Ovary (CHO) cells. CHO cells were transfected with pcDNA3 with cDNA for p80- or p130-angiomotin and stable cell lines were generated. A: Analysis of expression of angiomotin in CHO cell lines by western blot using the antibody against the C-terminus of angiomotin. Wt, wild type cells, Vec, vector transfected cells. B.: localisation of angiomotin in CHO cells was analysed by immunofluorescence microscopy. CHO cells were stained with the antibody against the C-terminus of angiomotin (green) and antibody against ZO-1. Overlay of the two images is shown (merge) with co-localisation appearing as yellow. P80-angiomotin localises to the cytoplasm and TJs whereas p130-angiomotin localises to F-actin fibres (arrow) as determined by staining with phalloidin. ZO-1 was recruited to p130-positive foci along stress fibres (arrowheads). Bar represents 10 μm.

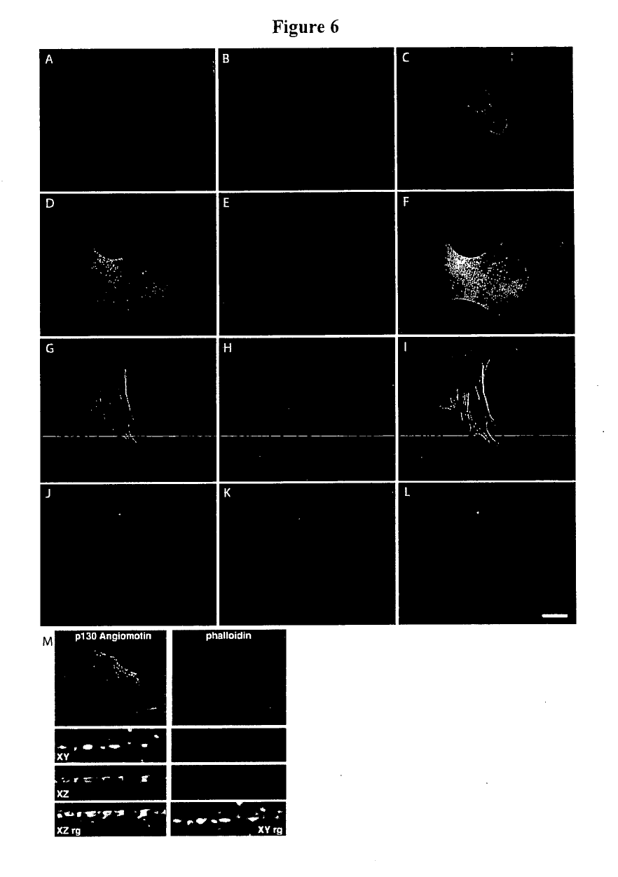

FIG. 6: The N-terminal domain of p130-angiomotin induces stress fibre formation. A-C: MAE cells retrofected with p80-angiomotin double stained with angiomotin rabbit polyclonal antibodies (green) and Texas-red conjugated phalloidin (red) showed that the p80 isoform localised to the lamellipodia of migrating cells. D-F: The same staining of MAE p130-angiomotin retrofected cells yields a different sub-cellular localisation. P130-angiomotin localised to stress fibres in a punctuate pattern. G-I: MAE cells transfected with the flag tagged construct encoding the N-terminal domain of p130-angiomotin were stained with a flag antibody and phalloidin. The flag antibody staining showed complete overlap with F-actin. J-L: MAE-vector cells stained with the angiomotin polyclonal antibodies and phalloidin. M: three dimensional imaging of p130-angiomotin and phalloidin staining showed co-localisation of p130-angiomotin and F-actin aggregates to the stress fibres. Scale bar: 20 μm.

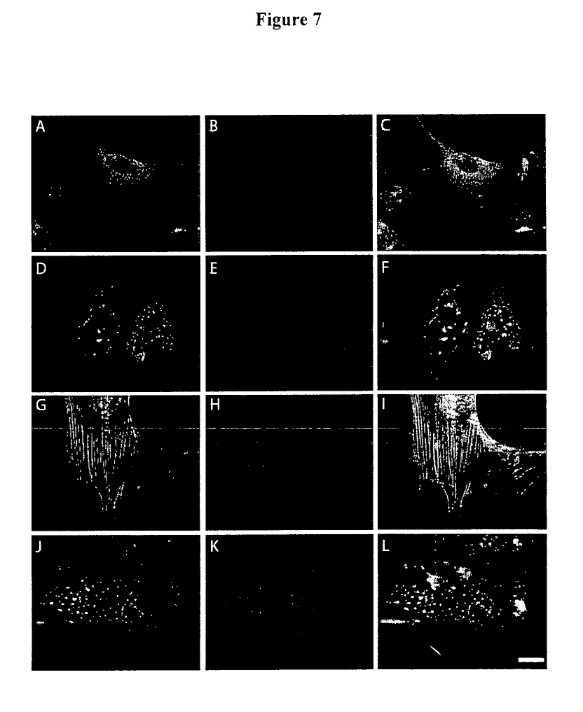

FIG. 7: p130-angiomotin remained associated with F-actin after disruption of stress fibres. A-C: MAE-p130-angiomotin cells stained for angiomotin (green) and F-actin (phalloidin, red). D-F same staining of MAE-p130-angiomotin cells after 1 hour of 50 μg/ml cytochalasin B treatment. Aggregated structures of p130-angiomotin entirely overlapped with the disrupted actin fibres. G-I:MAE cells transfected with the flag tagged N-terminus of p130-angiomotin stained with the flag antibody and phalloidin. J-L: same staining of the N-terminus transfected MAE cells 1 hour after addition of 50 μl/ml cytochalasin B. Aggregated structures of the N-terminus entirely overlapped with the disrupted actin fibres. Scale bar: 20 μm.

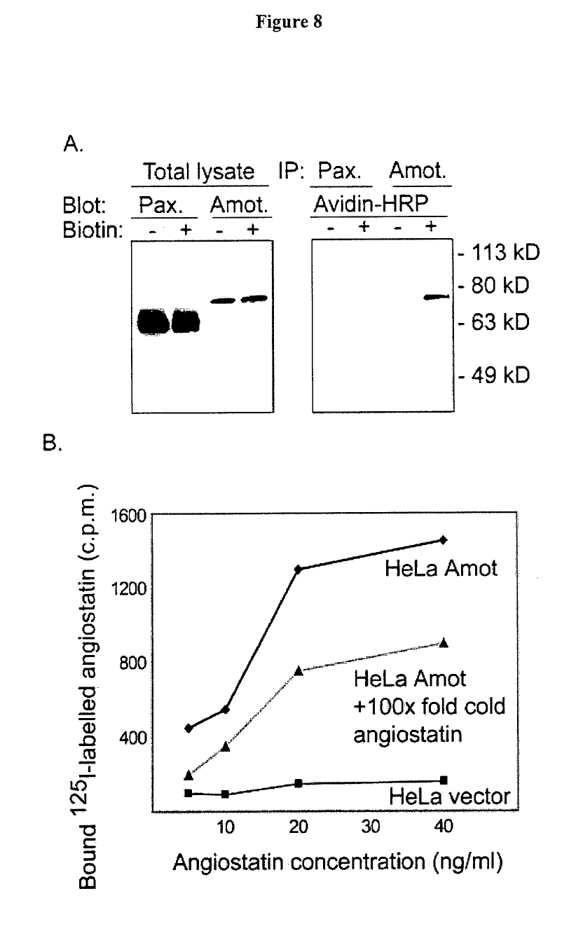

FIG. 8: Angiostatin binds angiomotin on the cell surface. A: Mouse aortic endothelial (MAE) cells stably expressing p80-angiomotin were incubated with Sulfo-NHS-LC-Biotin and subjected to immunoprecipitation (IP) with antibodies against the angiostatin binding domain of angiomotin or paxillin followed by blotting with HRP-conjugated avidin. Angiomotin was biotinylated whereas the intracellular protein paxillin was not. Input control lanes represent 2% of the amount that was used in the immunoprecipitation. B: binding of iodinated angiostatin to HeLa cells stably expressing p80-angiomotin. The binding was saturable with a half-maximal concentration of 11 ng/ml, or 0.3 nM.

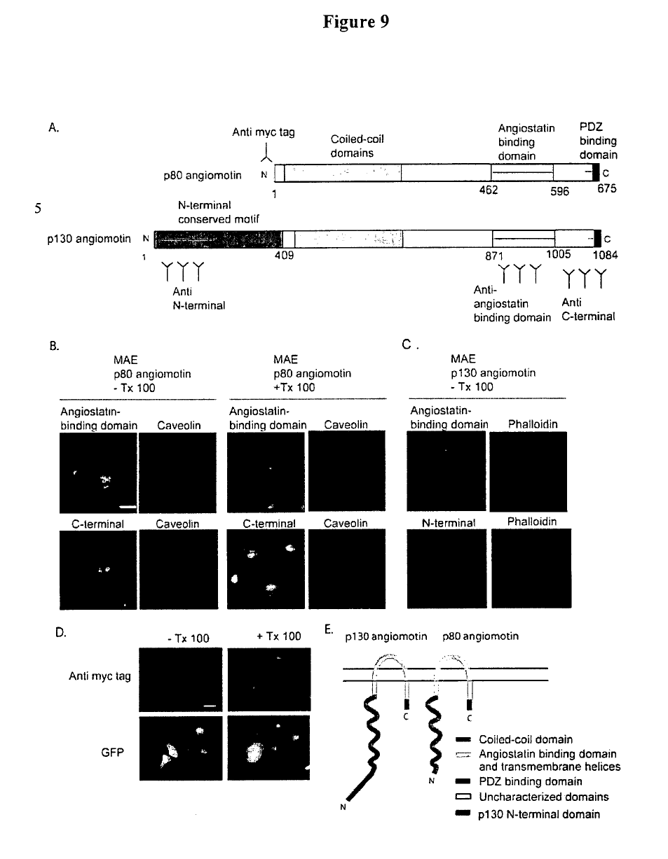

FIG. 9: The topology of angiomotin. A: The domain structure of p80- and p130-angiomotin. Three different polyclonal antibodies against angiomotin were used to probe the topology of angiomotin in MAE cells: one directed against the C-terminus, one directed against the angiostatin binding domain and one directed against the N-terminus of p130-angiomotin. The epitopes are shown as horizontal lines through the domains. These antibodies did not react with control cells expressing empty vector (not shown). In addition, myc antibody was used to localise p80-angiomotin tagged in the N-terminus. B: The antibody against the angiostatin binding domain (green) binds to p80-angiomotin on the cell surface. An antibody against an intracellular epitope of caveolin (red) was used as a control for an intact cell membrane. In contrast, the antibody against the C-terminus of angiomotin (green) only displayed immunoreactivity when cells were first treated with detergent. C: the antibody against the angiostatin binding domain (green) binds to p130-angiomotin on cells with an intact cell membrane. Here, phalloidin (red) was used as a control for an intact cell membrane. The antibody against the N-terminus of p130-angiomotin did not stain without permeabilisation. Vector cells did not stain with antibody. D: Cells expressing p80-angiomotin with a N-terminus myc-tag only stained permeabilised cells. The bar represents 20 μm. E: Suggested model for the topology of p80- and p130-angiomotin.

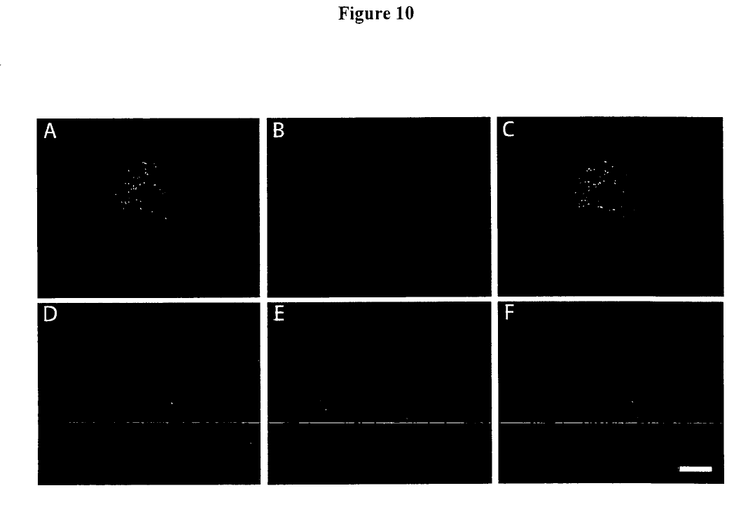

FIG. 10: The angiostatin binding domain of p130-angiomotin is exposed at the cell surface. A-C: MAE-P130-angiomotin cells stained with antibodies against the extracellular angiostatin binding domain without permeabilisation of the cell membrane showed staining of p130-angiomotin on the cell surface. Positive cells were negative for Texas-red-conjugated phalloidin staining thus showing that the cellular membrane was intact. D-F: Staining with the polyclonal antibodies towards the intracellular C-terminal and with phalloidin showed no positive staining for p130-angiomotin or for phalloidin. Scale bar: 20 μm.

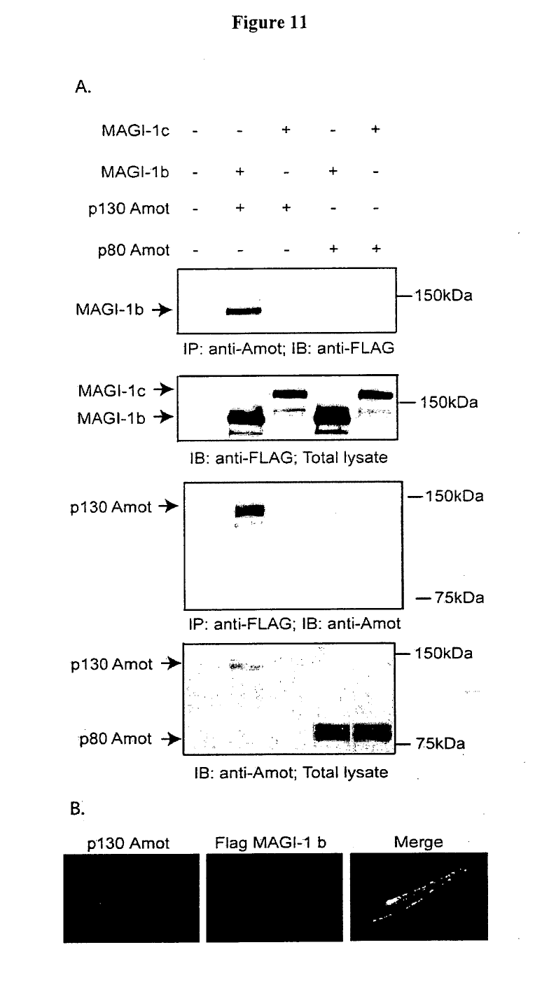

FIG. 11: p130-angiomotin interacts with MAGI-1. A: Lysates from CHO cells expressing p80 or p130-angiomotin and flag-tagged constructs of MAGI-1 B or MAGI-1 C were subjected to immunoprecipitation with flag antibody or the antibody against the C-terminus of angiomotin followed by immunoblotting with flag antibody or antibody against the C-terminus of angiomotin. Protein expression was confirmed by immunoblotting total lysate. B: p130-angiomotin co-localises with MAGI-1 B in CHO cells. CHO cells expressing p130-angiomotin and MAGI-1 B were immunostained with flag antibody (red) and antibody against the C-terminus of angiomotin (green). Overlay of the two images is shown (merge) with co-localisation appearing as yellow. Bar represents 10 μm.

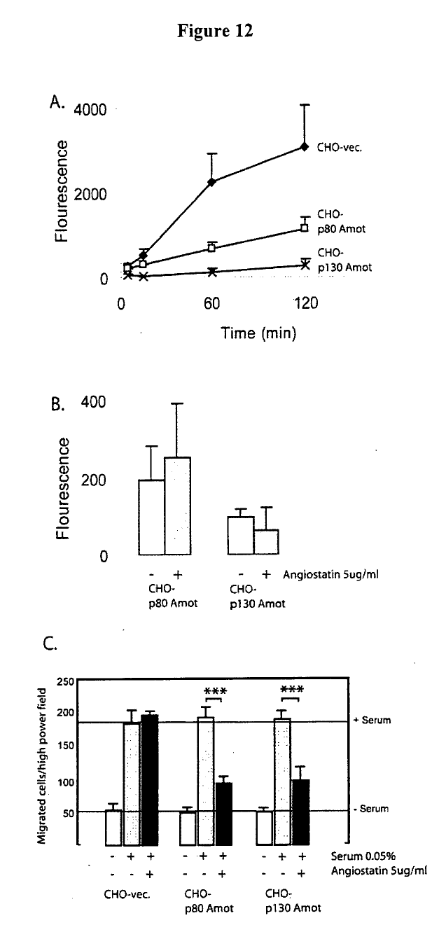

FIG. 12: Angiomotin controls permeability and migration. CHO cells stably expressing p80- or p130-angiomotin were analysed for their ability to control cell layer permeability and cell migration. A: Permeability of CHO cell monolayers grown in permeability chambers was measured as diffusion of FITC-dextran across a cell layer over time. Rhomboids, vector; squares, p80-angiomotin; crosses, p130-angiomotin. Means and standard error of the mean from one representative experiment with triplicates from each time point are shown. When data from three experiments were pooled, p<0.05 for vector-p80 and vector-p130 by students t-test. B: Angiostatin does not affect cell-permeability of angiomotin expressing CHO cells. Permeability of FITC-dextran was measured as in A, after 1 h. C: Angiostatin inhibits cell migration of angiomotin-expressing CHO cells. Cells were allowed to migrate spontaneously or towards 0.05% serum in a Boyden chamber. Standard deviation and the average number of migrated cells per microscopy field are shown.

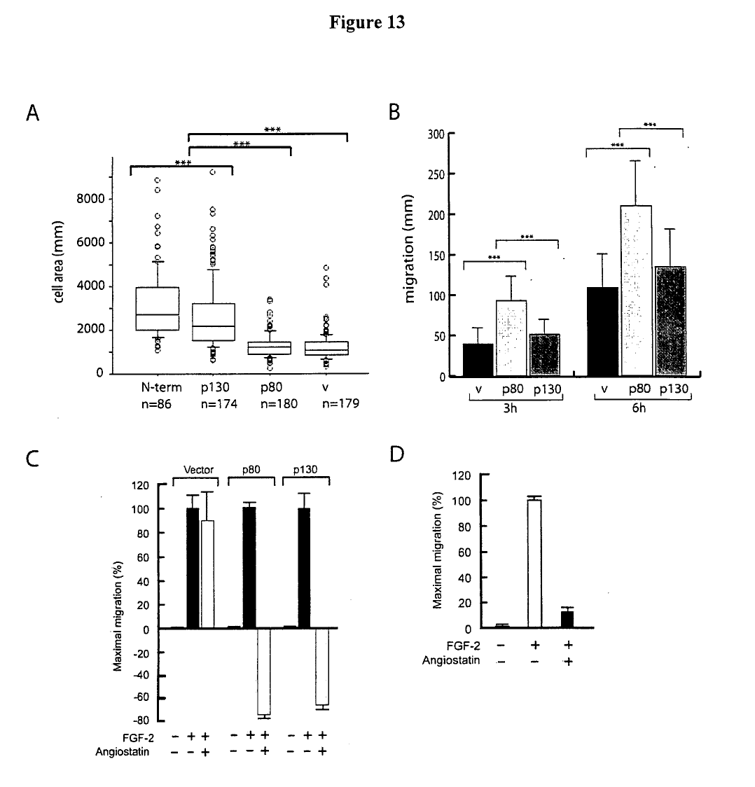

FIG. 13: p130-angiomotin affects cell shape and mediates angiostatin inhibition of migration. A: MAE-p130-angiomotin, MAE cells transfected with the N-terminal domain, MAE-p80-angiomotin cells and MAE-vector were stained and cell area was estimated as described in Materials and Methods. Cells expressing p130-angiomotin or its N-terminal fragment were more than two-times larger than p80-angiomotin and vector cells. Three stars indicate p<0.001. B: MAE cells expressing p130-angiomotin, p80-angiomotin and vector were grown to confluency and wounds were generated by scraping with a pipette. The migration rate was measured after 3 and 6 hours. P80-angiomotin cells migrated almost twice as fast as p130-angiomotin cells and vector cells. Three stars indicate p>0.001. C: MAE-p130-angiomotin, MAE-p80-angiomotin and MAE-vector cells were tested in the migration chamber assay. The cells were treated with or without bFGF and with or without angiostatin (5 μg/ml). The migration of vector cells was stimulated in the presence of bFGF but were not inhibited by angiostatin. The bFGF stimulated migration of p80-angiomotin was inhibited by 175% in the presence of angiostatin. The bFGF stimulated migration of p130-angiomotin was inhibited by 160% in the presence of angiostatin. D: hTERT⁺-BCE cells that endogenously express p80 and p130 angiomotin were analysed in the migration chamber assay. The migration was stimulated by bFGF and inhibited by 85% in the presence of angiostatin.

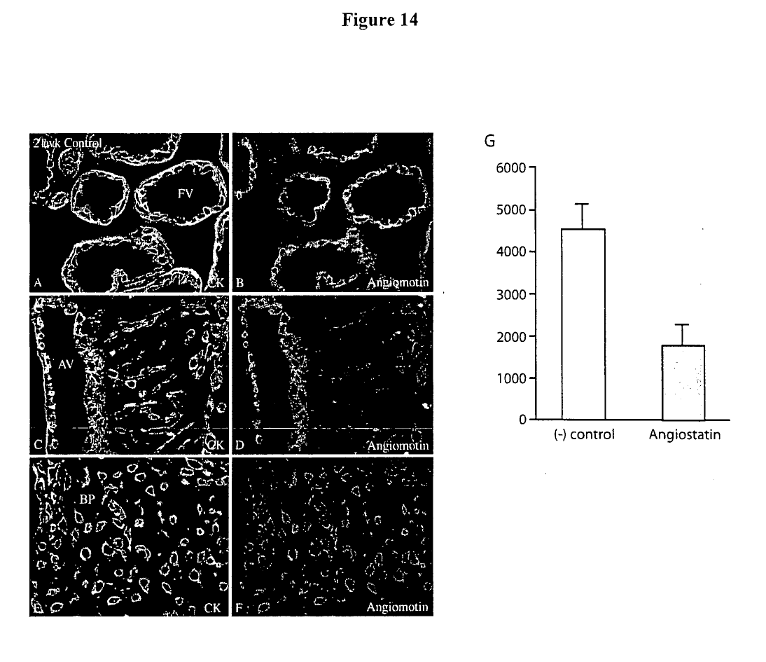

FIG. 14: Cytotrophoblasts express p80-angiomotin and p130-angiomotin and respond to angiostatin. A-F: Human placenta sections (second trimester) stained with polyclonal antibodies toward the C-terminal of angiomotin and an monoclonal antibody detecting cytokeratin (a marker for cytotrophoblasts) showed co-localisation of cytokeratin and angiomotin in floating villi (FV), anchoring villi (AV) and basal plate (BP). Magnification=400×. G: Angiostatin treatment (5 μg/ml) dramatically inhibited cytotrophoblast invasion of matrigel in vitro (p<0.001). The statistical significance of the data was analysed by Student's t-test.

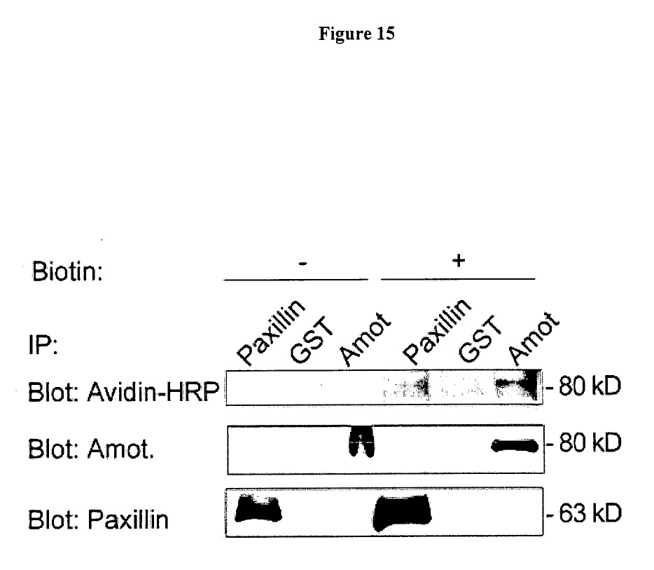

FIG. 15: Endogenous p80-angiomotin is biotinylated in Bovine Capillary Endothelial (BCE) cells. Similar experiments to those described in the legend to FIG. 8 were carried out in BCE cells to determine whether p80-angiomotin has any extracellular domains.

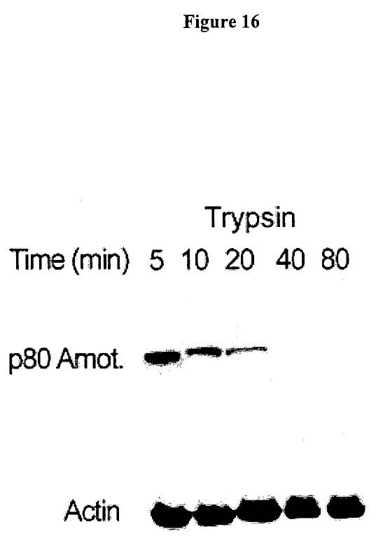

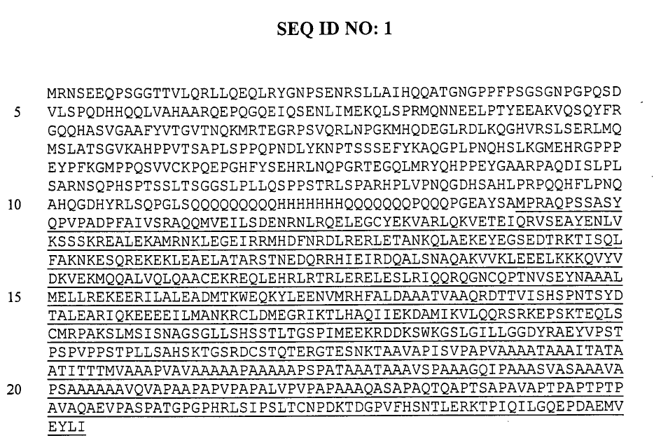

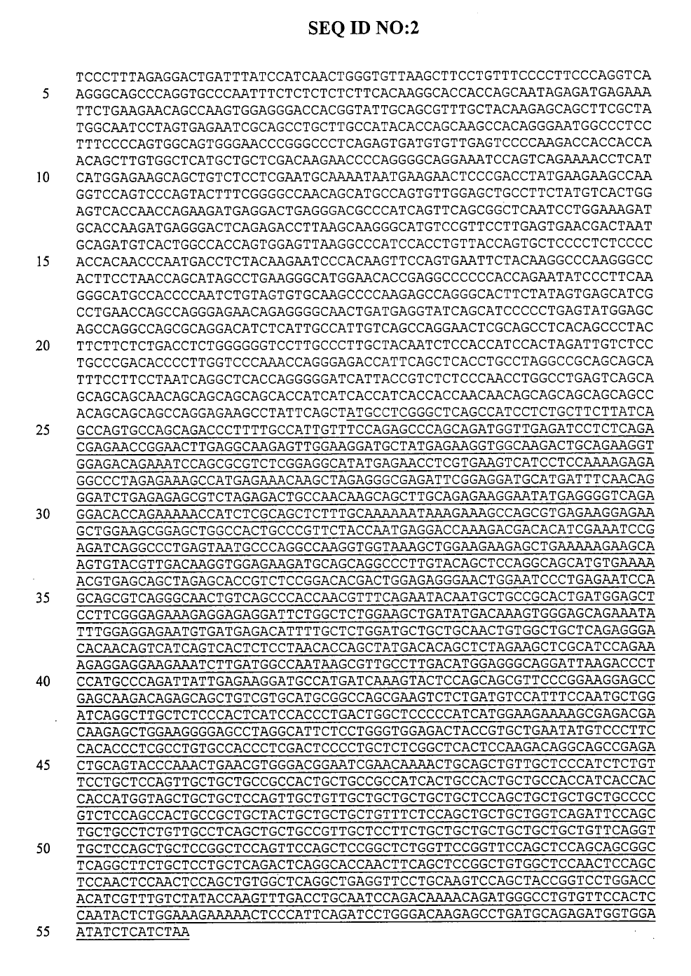

FIG. 16: p80-angiomotin has extracellular epitopes. MAE cells expressing p80-angiomotin (“p80 Amot”) were treated with trypsin for 5, 10, 20, 40 and 80 minutes and cell lysates analysed by Western blotting. Trypsin degraded most p80-angiomotin in 80 min, whereas actin, which is intracellular, was not degraded.

SEQ ID NO:1—Polypeptide sequence of p130-angiomotin. The p130-angiomotin N-terminal domain (which has no similarity with the sequence of p80-angiomotin) is not underlined; the p130-angiomotin sequence with similarity to p80-angiomotin is underlined.

SEQ ID NO:2—Polynucleotide sequence of p130-angiomotin. The polynucleotide sequence encoding the p130-angiomotin N-terminal domain (which has no similarity with the sequence of p80-angiomotin) is not underlined; the p130-angiomotin polynucleotide sequence with similarity to p80-angiomotin is underlined.

### EXAMPLE 1

### Experimental Data

### Materials and Methods

### Cell Culture

MAE cells stably expressing p80- and p130-angiomotin (Troyanovsky et al., 2001, J. Cell Biol., 152:1247-1254) were maintained in Dulbecco's modified Eagle's medium (DMEM) (Sigma). Chinese hamster ovary (CHO) cells were maintained in DMEM with F-12 HAM. CHO cells stably expressing p80 or p130 were generated by transfecting CHO cells with pcDNA3 with an insert of either p80-angiomotin or p130-angiomotin using Lipofectamine 2000 (Gibco) and selecting clones with G418 at 0.4 mg/ml. Bovine capillary endothelial (BCE) cells were cultured in DMEM supplemented with 2 ng bFGF/ml. All cell culture media was supplemented with 10% fetal calf serum (Gibco), 100 units/ml penicillin, 100 μg/ml streptomycin and 2 mM L-glutamine.

Spontaneously immortalized mouse aortic endothelial (MAE) cells (Bastaki et al., 1997, Arterioscler. Thromb. Vasc. Biol., 17:454-464) and ecotropic retrovirus producing Phoenix eco cells (provided by Dr G Nolan, Stanford University, Palo Alto, Calif.) were grown in Dulbecco's modified Eagle's medium (DMEM) (Sigma) with 10% fetal bovine serum, 1% glutamine and 1% penicillin/streptomycin.

Stably transfected MAE cells were grown in the presence of 5 g/ml puromycin (vector, p80-angiomotin, and p130-angiomotin) or 800 μg/ml G418 (p130-specific N-terminal). 293T cells were grown in DMEM medium supplemented with 10% donor bovine serum and antibiotics. M21 cell were grown in RPMI1640 medium containing 5-10% FCS and 2 mM Glutamine. BAE cells were grown in DMEM with 10% fetal bovine serum, 1% glutamine and 1% penicillin/streptomycin. hTERT⁺-BCE cells were grown in DMEM with 10% BCS, 2 ng/ml FGF-2, 1% glutamine and 1% penicillin/streptomycin.

### Plasmid Construction

The cDNA p80-angiomotin and p130-angiomotin were sub-cloned into the pENTR-2B vector (Gateway, Invitrogen) and then recombined into the convert Gateway destination vector pcDNA3 (Invitrogen) or pBABE (provided by Dr H Land, LCRF, London, UK) using the LR recombination reaction (Gateway, Invitrogen). Flag-tagged MAGI-1 b and MAGI-1 c were as described (Dobrosotskaya et al., 2000, Biochem. Biophys. Res. Commun., 270:903-9). The N-terminal construct was a kind gift from Dr A Shimono, Center for Developmental Biology, Kobe, Japan.

### Antibodies

Three different polyclonal antibodies against angiomotin were used: one against the angiostatin binding domain (B3 antibody) (Troyanovsky et al., 2001, J. Cell Biol., 152:1247-54), one against the C-terminus (TLE antibody) (Ernkvist et al., 2005, submitted) and one against the N-terminus of p130-angiomotin (Ernkvist et al., 2005, submitted) (FIG. 9A). The following mouse monoclonal antibodies were used: 9E10 anti-myc tag (Santa Cruz), M2 anti-flag tag (Sigma), AC-15 anti-actin (Sigma), 1A12 anti-ZO-1 (Zymed Laboratories Inc.), C060 anti-caveolin (Transduction Laboratories), 349 anti-paxillin (Transduction Laboratories) and rat monoclonal Mec 13.3 anti-CD31/PECAM (BD Pharmingen).

Rabbit polyclonal antibodies against different domains of angiomotin were generated. A fragment of the 266 amino acids specific for p130-angiomotin was used to produce the N-terminal antibody. The angiostatin-binding domain was used to generate antibodies against the extracellular part of angiomotin (Troyanovsky et al., 2001, J. Cell Biol., 152:1247-54). Peptides corresponding to the 24 most C-terminal amino acids were used to generate the C-terminal antibody.

### Immunoprecipitation

Cells were extracted with lysis buffer (50 mM, Tris-HCl pH 7.4, 0.2% Nonidet P40, 150 mM NaCl, 1 mM EDTA and protease inhibitors). Lysates were immunoprecipitated with either pre-immune or immune sera for 1 hour at 4° C. Pellets were washed 3 times with lysis buffer and resuspended in 1× sample buffer and analyzed by SDS-PAGE. For in gel digestion and mass spectrometry, 5 grams of 293T cells were solubilized in 50 ml of lysis buffer. The supernatant was clarified by centrifugation at 17,000 rpm for 20 minutes. 500 μl of a 50% slurry of pre-immune and immune anti-geminin antibodies coupled to sepharose CL-4B was used for immunoprecipitation at 4° C. for 90 minutes. The pellet was washed 3× with RIPA buffer (50 mM Tris pH 7.4, 150 mM NaCl, 1 mM EDTA, 1% NP40, 1% deoxycholic acid, 0.1% SDS and protease inhibitors). Samples were eluted with 100 mM ethanolamine for 2 minutes at room temperature, denatured with Lamelli sample buffer an analyzed on SDS-PAGE. Following silver staining, bands were excised and subjected to mass spectroscopy.

Cloning of p130-Angiomotin

Mass spectroscopy of proteolytic fragments from p130 sequence showed peptides that came from p80 and from what was then considered the 5′ “UTR” of p80-angiomotin (boldface in FIG. 1B) and from two ESTs in the data base (BI 058583 and CK001189.1). The extension matched a sequence in the genomic DNA, 7 kilobases upstream from the 5′ end of the p80 cDNA suggesting it was from an upstream exon. Because we were unable to acquire these ESTs, we used a PAC clone (AC004827.1) containing the genomic sequence; PCR amplified the upstream exon and ligated it to the 5′ end of the angiomotin p80 cDNA. The resulting cDNA contained the published sequence for ESTs B1058583 and CK001189.1 and the entire ORF of p130-angiomotin.

### Western Blot

Proteins were separated on 7.5% Criterion SDS-PAGE gels (Bio-Rad) and transferred to Protran nitrocellulose membranes by semi-dry blotting. Membranes where blocked by incubation with PBD with 5% and incubated with primary antibody at +4° C. overnight, followed by incubation with HRP-donkey anti rabbit or HRP-sheep anti mouse (Amersham Biosciences) for 1 h at RT. The filters were washed several times in PBS+0.05% tween and signal was visualized with Western Blotting Luminol Reagent (Santa Cruz).

Cell lysates were analyzed by SDS-PAGE and proteins were transferred to nitrocellulose membrane. Non-specific binding was blocked for 1 h in 10% dried milk in PBS containing 0.1% tween (PBS-T). The filter was incubated overnight at 4° C. in 5% dried milk in PBS-T with C-terminal peptide angiomotin polyclonal antibodies (1:500) or polyclonal antibodies towards the extracellular domain of angiomotin (1:2000). The secondary antibody (anti-rabbit-HRP) was diluted 1:10000 (Amersham) and incubated for 1 h at room temperature. Afterwards, the nitrocellulose membrane was visualized using a detection system from Santa Cruz Biotechnology Inc in an intelligent dark box (Fujifilm) with the program LAS1000 (Fujifilm). The membranes purchased from commercial sources that contained proteins from mouse adult tissues, embryos or placenta (RNWAY laboratories) were incubated as above.

### Biotinylation Experiments

Confluent 10 cm plates with approximately 10 million cells were briefly rinsed twice in PBS and incubated with NHS-Sulfo-LC-Biotin (Pierce Inc.) (0.4 mg/ml) in PBS or NHS-LC-biotin (0.4 mg/ml) in DMSO for 30 min at RT. The plates where then rinsed with PBS. Control plates where incubated with PBS alone. 1 ml of lysis buffer (20 mM HEPES, 140 mM KCl, 5 mM MgCl₂, 10 mM Beta-glycerophosphate, 3% polyethylene-9-lauryl ether (Thesit) and protease inhibitor cocktail, pH 7.4) was added and the cells were harvested using a rubber policeman. Lysates where spun at 30 000 g for 25 min and the supernatant were subjected to immunoprecipitation by incubation with either 1 μg of B3 angiomotin antibody or 1 μg of monoclonal anti-paxillin antibody and 30 μl of protein G sepharose slurry (Pierce Inc.). The beads were washed once in lysis buffer and three times in lysis buffer with 1% Thesit. Proteins were eluted with 30 μl of Laemli buffer and half of the material was loaded on a 10% precast Criterion gel (Bio-Rad). Biotinylated proteins were detected by western blot where membranes were incubated with HRP-conjugated streptavidin (Pierce Inc.) in PBS with 5% non-fat milk overnight.

### Angiostatin Binding Assay

Human angiostatin (kringle 1-4) was labelled with Iodine¹²⁵ by the Iodogen method according to the protocol of the manufacturer (Pierce Inc.). The specific activity was estimated at 15 000 cpm/ng protein. For binding assays HeLa cells stably expressing p80-angiomotin or empty vector was grown to confluency in 12-well plates. The cells were washed with PBS containing 1 mg/ml BSA. The cells were incubated with 10 ng/ml radio-labelled angiostatin for 2 h. Cells were then washed five times with PBS with 1 mg/ml BSA and lysed with 1% Triton X-100 in PBS and radioactivity was measured in a gamma counter.

### Trypsin Treatment

Confluent cells grown on 6 centimetre Petri dishes were washed twice with calcium- and magnesium free PBS and incubated with 1 ml sequence grade trypsin (Sigma) at 2 μg/ml or PBS alone at 37° C. for the indicated times. The experiment was ended by washing the cells once in PBS and addition of 75 μl Laemlli buffer. Samples were analysed by western blot.

### Bioinformatics Analysis

Transmembrane helices where predicted with PredictProtein (http://www.embl-heidelberg.de/predictprotein/predictprotein.html) (Rost et al., 1996, Protein Sci., 5:1704-1718) and Tmpred (http://www.ch.embnet.org/software/TMPRED_form.html).

### Immunofluorescence

Cultured cells were plated in chamber slides and allowed to grow and adhere overnight. The cells were fixed in 4% PFA for 10 min at room temperature. When needed, the cells were permeabilised on 0.1% triton X-100 (Sigma) for 1 min. Non-specific reactivity was blocked by incubating with 5% horse serum in PBS for 1 h before addition of primary antibody in blocking buffer for 1 h. antibody binding was detected with fluorescent-labelled secondary antibodies (Dako and Molecular Probes). F-actin was visualized with Texas-red phalloidin (Molecular Probes). The slides were mounted with mounting media from Vector Laboratories, viewed on a Zeiss Axioplan 2 microscope, and images collected using a AxioCam HRm Camera and the Axiovision 4.2 software. Cells treated with cytochalasin B (Sigma) were treated with 50 μg/ml cytochalasin B1 h prior to fixation. Then the cells were stained as described above.

Immunostaining of placenta sections: Sections of placenta were processed for double indirect immunolocalisation by using methods described previously (Fisher et al., 1989, J. Cell Biol., 109:891-902; Damsky et al., 1992, J. Clin. Invest., 89:210-222). Briefly, sections were fixed in cold acetone for 5 min, and then washed in PBS for another 5 min. Non-specific reactivity was blocked by incubation on 0.2% BSA/PBS for 30 min before addition of a mixture of primary antibodies. The rat anti-human cytokeratin monoclonal antibody 7D3 (Damsky et al., 1992, J. Clin. Invest., 89:210-222) was used at a dilution of 1:50. Rabbit anti-human angiomotin was used at a dilution 1:50. After 1 h, the cells were rinsed in PBS and incubated with the appropriate species-specific secondary antibodies (Jackson ImmunoResearch Lab, Inc) conjugated to rhodamine or fluorescine. After washing an additional three times in PBS, the coverslips were mounted on slides and examined with a Zeiss Axiophot epifluorescence microscope (Thornwood, N.Y.).

### Immunofluorescence of Cultured Cells

Cells plates on chamber slides (Falcon) were rinsed briefly in PBS, fixed in 4% para-formaldehyde for 10 min and (if not stated otherwise) treated with 0.05% Triton x-100 for 30 s. Cells were then incubated with 5% horse serum for 60 min, incubated with primary antibody diluted in 5% horse serum for 1 h, washed four times in PBS and incubated with Texas red horse anti mouse (Vector Laboratories Inc.) or FITC swine anti-rabbit (DAKO) diluted in 5% horse serum for 1 h. F-actin was visualized with Texas red phalloidin (Molecular Probes). Specimens where mounted in Vectashield mounting medium with DAPI (Vector Laboratories Inc.). Pictures where captured on a Ziess Axioplan 2 microscope and processed with Zeiss Axiovision software and Adobe Photoshop.

### Angiomotin Induction Assay

500 000 BCE cells were plated at the indicated densities and 24 h later cell layers where rinsed twice with PBS, briefly inverted on tissue paper, and lysed by addition of 100 μl 2×SDS-page loading buffer. Samples where analyzed by western blot using the TLE antibody. Sample volume increased with increasing plate size due to residual PBS, therefore, 10% of the volume of the lysates were loaded. Equal loading was verified by probing blots with an antibody against actin.

### Immunofluorescence of Mouse Retinas

Eyes from sacrificed C57BL6 mice were fixed in 4% para-formaldehyde/PBS at 4° C. for 2-3 h and washed in PBS. Retinas were dissected as previously described (Chan-Ling et al., 1990, Curr. Eye Res., 9:459-478) and incubated for 2 h at RT in a permeabilisation/blocking buffer, PBB:PBS containing 1% BSA, 0.5% Triton X-100 and 5% normal goat serum. Retinas were then incubated at 4° C. overnight with primary antibodies diluted in PBB buffer. After six washes with PBS at RT, retinas were incubated 2 h at RT in darkness with secondary antibody diluted in PBS+0.5% BSA, 0.25% Triton X-100 and 5% normal goat serum. The secondary antibodies were FITC-conjugated swine anti-rabbit (Dako) Alexa Fluor 594-goat anti-mouse (Molecular Probes) and RPE-goat anti-rat IgG mouse (Southern Biotechnology Associates). Retinas were flat mounted in Vectashield mounting medium (Vector labs).

Immunoprecipitation with MAGI-1

Two million CHO cells plated on 6 cm Petri dishes one day before were transfected with 2 μg of each plasmid DNA with Lipofectamine 2000 reagent (Life Technologies). Cells were harvested 48 h after transfection in a lysis buffer. 150 mM NaCl, 50 mM Tris-HCl pH 7.4, 0.5% Triton X-100 and protease inhibitors. The cell lysates were rotated end over end at 4° C. for 15 min and centrifuged at 13 000 rpm for 5 min at 4° C. The supernatants were collected and used for the determination of total protein by the Bradford method. Lysate representing 1.5 mg total protein were pre-cleared with 20 μl protein A beads and then incubated with 5 μg of anti-angiomotin or anti-FLAG antibody for each IP sample for 6 h at 4° C. under rotation. Afterwards, 30 μl of protein A beads were added to each IP sample and rotated overnight at 4° C. After incubation, beads were washed twice with lysis buffer, resuspended in Lammeli sample buffer, boiled and resolved by 7.5% Criterion precast gel (Bio-Rad). For immunofluorescence staining, 40 000 CHO cells were plated in chamber slides and transfected with 0.35 μg of each plasmid with Lipofectamine 2000 reagent.

### Aggregation Assay

CHO cells were de-attached by rinsing with calcium and magnesium free PBS twice and incubation with 0.02% EDTA (Sigma) until de-attached, resuspended in calcium and magnesium free Hanks balanced salt solution (HBSS) (Sigma), washed once in HBSS and resuspended to 100 000 cells/ml in HBSS supplemented with 2% FBS dialysed against calcium and magnesium free PBS. 50 000 cells were loaded per well in 24-well plates previously coated with 1% BSA. At this time the absolute majority of cells were single cells. CaCl₂ (2 mM) and angiostatin (5 μg/ml) was added where indicated. Cells were allowed to aggregate at 37° C. for 60 min during rotation on a platform rotator at 80 rpm. The experiment was stopped by addition of glutaraldehyde to a final concentration of 5%. At least five fields from each well were photographed at 10× magnification and analyzed for cell aggregation. The total number of cells (N0) were counted and the number of cell aggregates at 60 min (N60) were counted. Aggregation index was calculated as (N0-N60)/N0 as described (Hirata et al., 2001, J. Biol. Chem., 276:16223-16231). Approximately 500 cells where evaluated for each condition.

### Permeability Assay

The In Vitro Vascular Permeability assay kit from Chemicon Inc., which is based on the diffusion of FITC-labelled dextran across a cell layer grown on a membrane in a 24-well format, was used according to the manufacturer's instructions. Briefly, CHO cells were seeded at 10 000 cells per membrane insert and allowed to form a monolayer for five days. Triplicate or quadruplicate inserts were used for each condition. Where indicated, angiostatin (2 μg/ml) was added to the well one h before the start of the experiment. FITC-dextran was added to the upper chamber and 100 μl samples were withdrawn at 5, 15, 60 and 120 min from the lower chamber. Fluorescence was measured on a Bio-Tek FL 600 plate reader using an excitation wavelength of 485 nm and an emission wavelength of 530 nm. The background fluorescence of cell culture medium was subtracted. The diffusion of FITC-dextran across a membrane insert without cells was measured in parallel to ensure integrity of cell monolayers.

### Quantification of Cell Area

Cells were stained with the C-terminal angiomotin polyclonal antibodies for detection of positive cells and then stained with phalloidin to visualize the whole cell. Photos were taken with the AxioCam HRm camera attached to the Zeiss Axioplan 2 microscope. The cell areas were measured in Fujifilm image gauge version 4.0. Box plots and statistical calculations were done in Statview 5.0.1.

### In Vitro Wound-Healing

Cells were plated in chamber slides and grown overnight to confluency. A wound was made with a 200 μl pipette. Photos of the wounds were taken after 30 min (time point 0), 3.5 h (time point 3 h) and 6.5 h (time point 6 h). The distance between the cells in the wound edges were measured. Time points 3 h and 6 h were compared to time point 0 to show rate of wound closure. Statistical calculations were done in Statview 5.0.1.

### Migration Assay

Boyden chamber migration assay was performed as described (Troyanovsky et al., 2001, J. Cell Biol., 152:1247-1254). Briefly, 30 000 cells in serum free medium were loaded in each well and allowed to migrate towards 0.05% serum for four h. Non-migrating cells were removed and remaining cells were fixed and stained with Giemsa stain. Three fields per well were counted under a microscope.

Migration assays were performed in a modified Boyden chamber using a 48-well chemotaxis chamber (Neuroprobe Inc) as earlier described (Troyanovsky et al., 2001, J. Cell Biol., 152:1247-1254). Briefly, eight-micron Nucleopore polyvinylpirrolidine-free polycarbonate filters were hated with 100 μg/ml of collagen type 1 (Cohension, Palo Alta, USA) overnight. hTERT⁺-BCE, cells were starved in 0.2% FCS-DME medium for 16 hours. In order to test the inhibitory activity of angiostatin, hTERT⁺-BCE, and vector- and angiomotin MAE cells were pre-incubated with 5 μg/ml of angiostatin for 1 hour. The cells were trypsinised, resuspended in DMEM containing 0.1% bovine serum albumin (BSA) and 30,000 cells were added with or without angiostatin to each well of the upper chamber. Basic fibroblast growth factor (bFGF; Peprotech EC Ltd.) at 20 ng/ml was used as a chemoattractant in the lower chambers. The chemotaxis chambers were incubated for 3-5 hours at 37° C. with 10% CO₂ to allow cells to migrate through the collagen-coated polycarbonate filter. Non-migrated cells on the upper surface of the filter were removed and the filter was stained with Giemsa Stain (VWR International Ltd). The total number of migrated cells per field was counted at 20× magnification; each sample was tested in quadruplicates in three independent experiments.

### Placenta Tissue Collection

Informed consent was obtained from all patients from whom tissue was collected. Placental tissue from terminations of pregnancy (6 to 22 weeks) was collected within 1 hr of the procedure, washed repeatedly in PBS containing antibiotics, and placed on ice.

### Isolation of Cytotrophoblasts

Cells were isolated from pooled first- or second-trimester placentas by published methods (Fisher et al., 1989, J. Cell Biol., 109:891-902; Kliman et al., 1986, Endocrinology, 118:1567-1582). Briefly, placentas were subjected to a series of enzymatic digests, which detaches cytotrophoblast progenitors from the stromal cores of the chorionic villi. Once detached, the cells were purified over a Percoll gradient and cultured on Matrigel-coated substrates (Collaborative Biomedical Products) for 15 h in serum-free medium: DMEM, 4.5 g/L glucose (Sigma Chemical Co., St. Louis, Mo.) with 2% Nutridoma (Boehringer Mannheim Biochemicals), 1% penicillin/streptomycin, 1% sodium pyruvate, 1% Hepes and 1% gentamicin (UCSF Cell Culture Facility).

### Invasion Assay

Invasion assays were conducted as described previously (Damsky et al., 1992, J. Clin. Invest., 89:210-222; Librach et al., 1991, J. Cell Biol., 113:437-449). Briefly, isolated cytotrophoblasts (0.25×10⁶) were plated on Transwell inserts (6.5 mm, Costar) containing polycarbonate filters (pore size 8 μm) that had been coated with Matrigel. Experimental culture contained angiostatin added to the cultured medium at 5 μg/ml. After 48 hours the cultures were stained with the 7D3 antibody, which specifically reacts with human cytokeratin, to visualize the cytotrophoblasts. The filters were cut from the supports and mounted on slides with their upper surface facing down. The number of cytokeratin-positive cells and cell processes on the lower surface of filter were counted. Each experimental condition was tested in triplicate and the entire assay was performed three times.

The data were expressed as the total number of cytotrophoblasts and cytotrophoblast processes. The statistical significance of the data was analyzed by student's t-test.

### Results

### Identification of a Novel Splice Isoform of Angiomotin

During an immunoprecipitation experiment designed for other purposes we identified a novel isoform of angiomotin in 293T cells. The 293T cells were subjected to immunoprecipitation analysis and the immunoprecipitates were separated by SDS Polyacrylamide Electrophoresis (SDS PAGE) and detected by silver staining. Two bands corresponding to 80 and 130 kDa were cut out and identified by in-gel proteolysis and mass spectrometry. Sequencing of the tryptic digests from the two proteins identified the 80 kDa band and the 130 kDa protein band as an isoform of p80-angiomotin. Western blot analysis using an angiomotin-specific antibody confirmed that in 293T cells there are two isoforms of angiomotin (FIG. 1A).

In p130-angiomotin, amino acids 1-409 form the N-terminal part that is specific for p130-angiomotin and amino acids 410-1084 correspond to p80-angiomotin and form the C-terminal part of p130-angiomotin (FIG. 1B). P130-angiomotin is generated by alternative splicing between exons 2 and 3 that gives rise to this isoform with an N-terminal extension (FIG. 1C). We then generated a polyclonal antibody specific for the N-terminal domain of p130-angiomotin. This antibody specifically recognises the p130 and not the p80 isoform as analysed by Western blot (FIG. 1D).

### Expression of Angiomotin

### P130-Angiomotin is Expressed in the Placenta, But not in the Embryo Proper During Gestation.

Angiomotin plays an important role during embryogenesis, as angiomotin knock-out mice show abnormal visceral endoderm cell migration during gastrulation resulting in embryonic lethality in 70% of the mice (Shimono and Behringer, 2003, Current Biol., 13:613-7). We therefore analysed the temporal expression pattern of the angiomotin isoforms during mouse embryogenesis. A Western blot of mouse embryos from embryonic day (E) 5 to 19 showed expression of p80-angiomotin from E6 with an increase of protein level during fetal development. No expression of p130-angiomotin was detected (FIG. 2A). Overexposure of embryonic lysates from E15 showed a weak expression of p130-angiomotin (data not shown). Therefore, we concluded that p80-angiomotin is the dominant form expressed during embryogenesis. We then analysed angiomotin expression in extra-embryonic tissues using a Western blot approach with placenta samples from different stages of gestation. In addition to p80-angiomotin that was expressed from E11 to the end of gestation, p130-angiomotin expression was only detected in the placenta from E13 to E16 (FIG. 2B). Analysis of angiomotin protein levels in adult mouse tissues showed that p130-angiomotin expression was restricted to the thymus and testis whereas p80-angiomotin was more abundantly expressed in the different adult tissues (FIG. 2C).

To determine the expression of p130-angiomotin and p80-angiomotin at a cellular level, we tested cell lysates from 293T cells, cytotrophoblasts, hTERT⁺-BCE cells and bovine aortic endothelial (BAE) cells. As shown in FIG. 2D all cell types tested produce both isoforms of angiomotin.

### Localisation of Angiomotin

### Angiomotin Localizes to Tight Junctions in Primary Endothelial Cells and is Controlled by Cell Density

In order to verify the extracellular staining pattern in primary cells we carried out the same experiments with BCE cells which express p80- and p130-angiomotin in similar amounts (Ernkvist et al., 2005, submitted). Without using membrane permeabilisation, the antibody against the angiostatin binding domain stained approximately 10% of the cells with a pattern similar to that of MAE cells. A control antibody against GST that was used as a negative control did not stain (data not shown). We proceeded to investigate the localization of angiomotin in confluent primary cells. Surprisingly, the antibody against the angiostatin binding domain showed no detectable staining on confluent cells in the absence of detergent, suggesting that the angiostatin binding domain is masked when cells are confluent (FIG. 3A). However, the antibody against the C-terminus of angiomotin displayed strong immuoreactivity in cell-cell contacts. The localization overlapped with ZO-1, a marker for tight junctions (TJs). Angiomotin could also be detected in what appeared to be an intracellular pool, occasionally positive for ZO-1. This, together with the observation that, when the antibody against the angiostatin binding domain was used only 10% of sub-confluent cells displayed surface immunoreactivity, and that completely confluent cells displayed no immunoreactivity, suggested that angiomotin is recruited to the cell surface at the place of cell-cell contacts in these cells. Treatment of confluent cells with angiostatin (5 μg/ml) did not alter the localisation of angiomotin (data not shown).

The fluorescent staining appeared stronger when BCE cells were more confluent. In order to investigate this we plated BCE cells at different densities and analyzed angiomotin expression by western blot. Expression of both p80- and p130-isoforms of angiomotin were strongly induced at 25 000 cells/cm² when cells could form contacts with each other (FIG. 3C). We concluded that expression of angiomotin expression is regulated by formation of cell-cell contacts.

### Angiomotin Localizes to Cell-Cell Contacts in Endothelial Cells In Vivo

We proceeded to analyse the location of angiomotin in endothelial cells in vivo. In the retina of the mouse, angiogenesis occurs during postnatal days (P) 1 to 14 as vessels sprout from a vessel in the optic nerve towards the periphery of the retina and subsequently into deeper layers. We performed whole mount staining of retinal vessels from mice at postnatal days P5 and analyzed the expression of angiomotin by immunofluorescence. Angiomotin was expressed in endothelial cells together with CD31/PECAM, a marker for endothelial cells (FIG. 4A). This shows that angiomotin expression is specific for endothelial cells in the retina. The angiomotin staining overlapped with ZO-1 signal showing that angiomotin is localized to cell-cell contacts in endothelial cells in vivo (FIGS. 4B and C).

### Angiomotin Localizes to Tight Junctions and Recruits ZO-1 in CHO Cells

We used CHO cells, which are often used as a model system for cell-cell contacts, to investigate the functionality of angiomotin in TJs. We made stable lines of CHO cells expressing p80-angiomotin, p130-angiomotin or empty vector. Transfection led to the appearance of bands of the expected molecular weights in western blot analysis (FIG. 5A). However, in p130-angiomotin transfected cells additional bands appeared at 120 kDa and 80 kDa indicating that additional in-frame ATGs in the p130 mRNA can serve as translation initiation sites as previously reported (Ernkvist et al., 2005, submitted). CHO p130 clones expressed protein at somewhat lower amounts than p80 clones. Wild type cells and cells transfected with empty vector expressed no detectable amount of protein.

We analyzed the localization of angiomotin in CHO cells by immunofluorescence. P130-angiomotin was localized to cell-cell whereas p80 was localized to the cytoplasm as well as TJs. In cell-cell contacts the angiomotin staining overlapped with ZO-1 (FIG. 5B) which shows that angiomotin co-localize with ZO-1 (FIG. 5B) which shows that angiomotin co-localise with ZO-1. Occasionally, p130 was localized in foci along F-actin stress fibres that stained with phalloidin, as reported before (17). ZO-1 could be recruited to these foci in manner not seen in control cells (FIG. 5B), which indicates that angiomotin can control the localization of ZO-1.

N-Terminal Domain of p130-Angiomotin Localises to Actin and Stabilises Stress Fibres.

As we have previously reported, p80-angiomotin localises to the lamellipodia (FIG. 6A-C) (Troyanovsky et al., 2001, J. Cell. Biol., 152:1247-1254). To compare the sub-cellular localisation of p130-angiomotin and p80-angiomotin, mouse aortic endothelial cells (MAE) were retrofected with constructs encoding the individual isoforms. Polyclonal positive cells were selected with puromycin and expression was detected by Western blot analysis (FIG. 2D). The cells retrofected with p130-angiomotin showed a 130 kDa band but also a weak band at 80 kDa, indicating that the p130-angiomotin open reading frame (ORF) may produce both isoforms. The MAE cells retrofected with these proteins were stained with the C-terminal-specific antibody and the staining of p130-angiomotin positive cells showed a very regular pattern of small linear dots (FIG. 6D). This staining pattern was not restricted to MAE cells as M21melanoma and 293T cells showed similar structures when transfected with p130-angiomotin (data not shown). The filamentous expression pattern of p130-angiomotin suggested that p130-angiomotin may localise to F-actin. To assess potential actin association, MAE cells expressing p130-angiomotin were stained for F-actin with Texas-red conjugated phalloidin as well as for p130-angiomotin. The punctuated p130-angiomotin staining showed a direct overlap with F-actin in most locations (FIG. 6D-F). An interesting observation from the stainings is that vector cells (FIG. 6J-L) and p80-angiomotin cells do not have the same actin fibre pattern as p130-angiomotin. P130-angiomotin induces stress fibres whereas vector and p80-angiomotin expressing cells have less actin fibres. This indicates that p130-angiomotin stabilises stress fibres.

To further analyse the co-localisation of p130-angiomotin and F-actin, we used computerised deconvolution together with a 3D imaging software to determine the overlap of p130-angiomotin and F-actin. FIG. 3M shows the co-localisation of angiomotin and actin in one single stress fibre.

Next we investigated whether the N-terminal portion that is unique to p130-angiomotin is mediating the association with actin. For this purpose, MAE cells were transiently transfected with the N-terminal domain that is specific to p130-angiomotin. Immunofluorescent staining of the N-terminal fragment showed a perfect co-localisation with stress fibres but lacked the punctuated pattern characteristic of the full-length p130-angiomotin (FIG. 6G-I). Similar staining patterns were also observed in HeLa and M21 cells transfected with the N-terminal domain (data not shown). These results show that the 409 amino acids in the N-terminal of p130-angiomotin associate with stress fibres.

Similar to the p130-angiomotin transfected cells, the N-terminal transfected cells had more stress fibres that vector and p80-angiomotin cells, indicating that the N-terminus of p130-angiomotin induces a more stress fibre-like pattern.

To investigate whether p130-angiomotin is associated with F-actin, we treated the cells with the actin depolymerising agent cytochalasin B. As shown in FIG. 7D, the punctuated staining pattern of p130-angiomotin was disrupted in the cells treated with cytochalasin B. The disrupted p130-angiomotin staining overlapped entirely with the phalloidin staining of aggregated structures of actin (FIG. 7D-F). Next, we transfected the N-terminal domain of p130-angiomotin into MAE cells (FIG. 7G-I) and treated these cells with the cytochalasin B. Again, the N-terminal staining overlapped entirely with the aggregated structures of actin (FIG. 7J-L). To ensure that the staining was not due to a cell collapse, we also stained for tubulin and cytochrome C. Stainings of neither tubulin nor cytochrome C was affected by cytochalasin B treatment (data not shown). We also treated p80-angiomotin transfected MAE cells with cytochalasin B. The lamellipodia staining of p80-angiomotin was disrupted, but the intracellular staining detected in all p80-angiomotin positive cells was not affected (data not shown).

### Topology of Angiomotin

### Angiostatin Binds Angiomotin on the Cell Surface

We have previously reported that Mouse Aortic Endothelial cells (MAE cells) transfected with p80-angiomotin respond to angiostatin by blocking migration and tube formation in vitro. This suggests that p80-angiomotin is a receptor for angiostatin. However, as judged by sequence analysis, angiomotin does not have an obvious signal peptide that could mediate the insertion of the protein into the membrane. In order to investigate whether p80-angiomotin has any extracellular domains we incubated MAE cells with Sulfo-NHS-LC-biotin, a biotin derivative with a reactive group that conjugates biotin to proteins. Sulfo-NHS-LC-biotin is water soluble and will not penetrate intact cell membranes. After incubation with Sulfo-NHS-LC-biotin we subjected cell lysates to immunoprecipitation against either angiomotin, or, as a negative control, the intracellular protein paxillin. The immunoprecipitates were analyzed by western blot with HRP-conjugated avidin. As seen in FIG. 8A, p80-angiomotin was biotinylated whereas paxillin was not. Paxillin could, however, be biotinylated with a hydrophobic, membrane-permeating analogue, NHS-LC-biotin (data not shown). Endogenous p80-angiomotin could also be biotinylated in primary cells when similar experiments were carried out with bovine capillary endothelial (BCE) cells (FIG. 15). This is of interest as angiostatin was first identified by its ability to inhibit proliferation of these cells (O'Reilly et al., 1994, Cell, 79:315-328). In order to verify that angiomotin has extracellular epitopes we treated MAE cells with trypsin for various times, and analyzed cell lysates by western blotting. Trypsin degraded most p80-angiomotin in 80 min, whereas actin, which is intracellular, was not (FIG. 16).

Previously we have shown that angiomotin transfected HeLa cells can bind and internalize FITC-labelled angiostatin (Troyanovsky et al., 2001, J. Cell Biol., 152:1247-54). In order to verify the binding of angiostatin to angiomotin-expressing cells, HeLa cells stably expressing angiomotin were incubated with iodinated angiostatin at different concentrations, and scintillation was measured (FIG. 8B). Angiostatin bound to these cells in a saturable way with a half-maximal concentration of 11 ng/ml, or 0.3 nM, which can be considered an approximation of Kd. Angiostatin did not bind to control cells to any great extent. A one hundred fold concentration of cold angiostatin could compete with the binding of labelled angiostatin. However, it was not feasible to add angiostatin in an amount that would completely block the binding of labelled angiostatin as a thousand fold concentration would be needed. In conclusion, our data show that angiomotin is localised on the cell surface where it binds angiostatin.

### Topology of Angiomotin

Next, we proceeded to analyse the topology of angiomotin. By sequence analysis, p80-angiomotin contains three distinct domains. The N-terminus half is predicted to form a coiled-coil (Bratt et al., 2002, Gene, 298:69-77) and the C-terminus has a putative PDZ-binding domain which is important for controlling cell motility (Levchenko et al., 2003, J. Cell Sci., 116:3803-3810). The angiostatin binding domain identified in the yeast two-hybrid screen is a partly hydrophobic domain of 135 residues (Troyanovsky et al., 2001, J. Cell Biol., 152:1247-54) located in the central region of the polypeptide (FIG. 9A). p130-angiomotin contains an extended N-terminal domain with conserved glutamine rich motifs. Bioinformatics analysis suggests that angiomotin contains up to 3 putative transmembrane helices in the hydrophobic part of the angiostatin binding region (data not shown). We hypothesized that the angiostatin binding domain is extracellular and that the N-terminal coiled-coil as well as the PDZ binding domains were intracellular.

Generally, the cell membrane must be permeabilised for antibodies to stain intracellular epitopes in immunofluorescence studies. To investigate the transmembrane topology of angiomotin, immunofluorescence staining of sub-confluent cells with antibodies directed against different domains of angiomotin in the absence or presence of prior treatment with the detergent Triton X-100 was used. We used three different polyclonal rabbit antibodies: one that is directed against the angiostatin binding domain (Troyanovsky et al., 2001, J. Cell Biol., 152:1247-54), one directed against the most C-terminal 24 residues, and one directed against the N-terminus of p130-angiomotin (FIG. 9A). The antibody directed against the angiostatin binding domain could stain without prior extraction of the membrane, suggesting that this domain has extracellular epitopes (FIG. 9B). As a control for an intact cell membrane we stained for the intracellular N-terminal domain of caveolin, which only stained cells treated with Triton X-100. In contrast, the antibody directed against the C-terminus of angiomotin needed permeabilisation in order to stain (FIG. 9B). When Triton X-100 was used, this antibody stained the cells in a similar staining pattern as the antibody against the angiostatin binding domain. This indicates that the C-terminus is intracellular, although it can not formally be ruled out that this epitope is extracellular and becomes available for the antibody after the detergent removes a masking protein which is bound to the c-terminus. However, the fact that this epitope contains the PDZ binding domain and such domains, as far as is known, are always intracellular, argues for the former conclusion. By the same methodology, the antibody against the N-terminus of p130-angiomotin also only displayed immunoreactivity when cells were first permeabilised, indicating that this domain is intracellular (FIG. 9C). Finally, in order to analyze the N-terminus of p80 angiomotin in the same manner we transfected MAE cells with a construct for angiomotin with a myc-tag in the N-terminus. We co-transfected with a green fluorescent protein (GFP) plasmid as a transfection marker. Staining for the N-terminal myc-tag required permeabilisation (FIG. 9E), indicating that the N-terminus of angiomotin is intracellular. In conclusion, these data indicate that the N-terminus and the C-terminus of angiomotin are intracellular and the hydrophobic region flanking the angiostatin binding domain can form two transmembrane helices leaving the central part of the angiostatin binding in the extracellular space.

The Angiostatin Binding Motif of p130-Angiomotin is Exposed at the Cell Surface

As shown above and in previous studies (Troyanovsky et al., 2001, J. Cell. Biol., 152:1247-1254; Bratt et al., 2005, submitted), p80-angiomotin contains an extracellular angiostatin-binding domain. We therefore analysed whether the angiostatin-binding motif is exposed at the cell surface in p130-angiomotin expressing cells. Non-permeabilised MAE cells expressing p130-angiomotin were stained with the polyclonal antibody directed against the extracellular angiostatin-binding domain. The cells were also incubated with phalloidin to verify that the outer membrane of the cells were intact. The same punctuated pattern of p130-angiomotin could be observed in the non-permeabilised cells that were negative for phalloidin staining (FIG. 10A-C). No staining of p130-angiomotin could be detected in cells stained with the polyclonal antibodies directed against the intracellular C-terminal domain of angiomotin (FIG. 10D-F). These results indicate that p130-angiomotin also contains an extracellular angiostatin binding domain as p80-angiomotin does.

### Function of Angiomotin

Angiomotin Interacts with MAGI-1

The co-localization of angiomotin with ZO-1 prompted us to attempt to co-immunoprecipitate angiomotin and ZO-1. However, no such interaction could be found. Neither could be establish an interaction between occluding and angiomotin. We then turned our attention to MAGI-1, a membrane-associated guanlyate kinase (MAGUK) related to ZO-1, which has been reported to bind to the cytoplasmic domain of endothelial cell-selective adhesion molecule (ESAM) in endothelial cells (Wegmann et al., 2004, Exp. Cell Res., 300:121-133). In CHO cells transiently expressing p80 or p130 angiomotin and flag tagged MAGI-1 isoforms MAGI-1b and MAGI-1c, MAGI-1b, but not MAGI-1c, could be immunoprecipitated with p130-angiomotin but not with p80-angiomotin. Also, p130-, but not p80-angiomotin, could also be immunoprecipitated with flag antibody (FIG. 11A). Immunofluorescence studies revealed that p130 and MAGI-1b co-localized when expressed in CHO cells (FIG. 11B). Thus, the N-terminal domain of p130-angiomotin can associate with MAGI-1b or with proteins that interact with MAGI-1b.

### Angiomotin Controls Cell Migration and Permeability

In order to analyze the function of angiomotin in cell-cell contacts we carried out cell aggregation assays. These indicated that angiomotin did not mediate cell aggregation in the absence or presence of calcium in a short-term aggregation assay and angiostatin did not have an effect on the aggregation of angiomotin-transfected cells (data not shown). We proceeded to investigate the role of angiomotin in controlling permeability of TJs. For this purpose we used an in vitro permeability assay where diffusion of FITC-labelled dextran across a layer of cells grown on a permeable membrane was measured. Fluorescence increased in a time dependant manner, but markedly slower in angiomotin expressing cells compared to control cells. After one hour p130-angiomotin expressing cells displayed 88% lower permeability compared to control cells and p80-angiomotin cell displayed 70% lower permeability (FIG. 12A). This shows that angiomotin not only localizes to TJs, but can itself affect TJ function. Angiostatin, however, did not affect permeability in either p80- or p130-angiomotin cells (FIG. 12B). MAE cells stably expressing p80-angiomotin respond to angiostatin by reduced migration in the Boyden chamber assay, whereas MAE control cells do not respond (Troyanovsky et al., 2001, J. Cell Biol., 152:1247-54). In order to investigate if CHO cells behave in a similar way, we performed Boyden chamber experiments with CHO cells expressing p80- or p130-angiomotin. The results are shown in FIG. 12C. CHO cells expressing p80- or p130-angiomotin responded to angiostatin with a 50% reduction of motility. Thus, the effect of angiostatin is limited to inhibiting cell migration and angiostatin does not affect angiomotin-mediated control of TJs.

### P130-Angiomotin Affects Cell Shape

The p130-angiomotin retrofected MAE cells contained more stress fibres and displayed a flattened morphology as compared to vector and p80-angiomotin expressing cells. The cells were stained for angiomotin expression together with phalloidin to visualise the outline of the cells. Cells that were positive for angiomotin staining were photographed and the cellular area was measured as described in the materials and methods. The median area of the MAE cells expressing p130-angiomotin or the N-terminal domain was more than two times larger than the p80-angiomotin or the vector cells as shown in the box plot diagram in FIG. 13A. These results show that p130-angiomotin regulates cytoskeleton organisation and cell shape, and it is the N-terminal part of the protein that mediates these effects.

### P130-Angiomotin is Inhibited by Angiostatin

Our previous studies have shown that angiomotin promotes migration of transfected cells. We therefore used an in vitro wound-healing assay to investigate the effect of p130-angiomotin expression on the migrating rate of MAE cells. In this assay, MAE cells transfected with p80-angiomotin, p130-angiomotin or vector were grown to confluence and wounds were subsequently generated by scraping with a pipette. The migration rate was estimated by analysing the distance of the leading edge of the migrating cells from the edge of the wound. The results from the wound-healing demonstrated that expression of p80-angiomotin stimulated migration towards the wound almost two-fold whereas vector cells and p130-angiomotin cells had similar migrating rate (FIG. 13B). Next we analysed whether MAE cells expressing p130-angiomotin respond to angiostatin. First we examined if angiostatin could affect the cell shape of the p130-angiomotin positive cells. The cells were incubated with 5 μg/ml angiostatin overnight and were fixed and analysed the following day. The results showed that angiostatin treatment did not significantly affect the cell area of p130-angiomotin cells (data not shown).

We have previously shown that migration of MAE cells expressing p80-angiomotin is inhibited by angiostatin in the Boyden chamber assay (Troyanovsky et al., 2001, J. Cell. Biol., 152:1247-1254). We therefore compared the migration of p130-angiomotin, p80-angiomotin, and vector cells in the presence or absence of angiostatin. The migration of vector transfected cells was stimulated in the presence of bFGF but was not inhibited in the presence of angiostatin. The bFGF-stimulated migration of both p130-angiomotin and p80-angiomotin was inhibited by 160 and 175 percentages respectively in the presence of angiostatin, which is below the level of migration in the absence of bFGF (FIG. 13C).

Since hTERT⁺-BCE cells express p80-angiomotin and p130-angiomotin endogenously, we tested them in the migration chamber assay. The migration of hTERT⁺-BCE cells was simulated in the presence of bFGF and inhibited by 85% when treated with angiostatin (FIG. 13D). Our results are in line with previous reports showing that angiostatin inhibits endothelial cell migration (Claesson-Welsh et al., 1998, Proc. Natl. Acad. Sci. USA, 95:5579-5583).

### Angiostatin Inhibits the Invasion of Cytotrophoblasts

Cytotrophoblasts are specialised epithelial cells of the human placenta that differentiate to acquire tumour-like properties that allow them to invade the uterus where they develop endothelial-like characteristics (reviewed in Red-Horse et al., 2004, J. Clin. Invest., 114:744-754). This is indicated by their expression of PECAM, VE-cadherin, VCAM-1, αv and αvβ3 integrin. Previously we detected expression of angiomotin only in endothelial cells. However, as human cytotrophoblasts also express the p130-angiomotin and p80-angiomotin isoforms (FIG. 2D) we stained placenta sections. As shown in FIG. 14, the staining showed co-localisation of cytokeratin and angiomotin in floating villi, anchoring villi and basal plate, indicating that both p80-angiomotin and p130-angiomotin are expressed in all the cytotrophoblasts populations (e.g. progenitor, invasive) and not in the differentiated syncytiotrophoblasts (FIG. 14A-F).

Next we tested whether invasion of cytotrophoblasts, which express high amounts of endogenous p130-angiomotin and p80-angiomotin, was affected by angiostatin treatment. The invasion assays were performed in the presence or absence of angiostatin. After 48 hours, the invading cells were stained with a cytokeratin monoclonal antibody and the experiment scored as described in Materials and Methods. The result showed that in the experiments where angiostatin were added, the cytotrophoblasts invasion was down-regulated almost three-fold as compared to the control (FIG. 14G).

### Discussion

We have identified a novel splice isoform of angiomotin that regulates cell shape and mediates angiostatin inhibition of endothelial cell migration. p130-angiomotin contains an extended amino terminal domain but is otherwise identical to its shorter isoform of 80 kDa. The first 243 amino acids of the N-terminal domain show 58% homology to Amot1-1 and Amot1-2. within these 243 amino acids there are five islands of conserved regions rich in glutamine consisting of about 8-10 amino acids which are 100% conserved between the three proteins. Little is known regarding the cellular localization and function of Amot1-2 but Amot1-1 (JEAP) has been shown to be specifically expressed in exocrine cells and localize to ZO-1 in tight junctions in vitro and in vivo (Nishimura et al., 2002, J. Biol. Chem., 277:5583-7). The sub-cellular localization of p130-angiomotin was distinct from p80-angiomotin in MAE-cells. In contrast to the lamellipodia staining observed with p80-angiomotin, p130-angiomotin was expressed in a regular punctuated pattern overlapping with F-actin. This staining pattern did not overlap with that of tensin found in fibrillar adhesions (Zamir et al., 1999, J. Cell Sci., 112:1655-1669) or α-actinin, an actin-binding protein (Belkin and Koteliansky, 1987, FEBS Letts, 220:291-4; Otey et al., 1990, J. Cell Biol., 111:721-9) (data not shown).

Cells expressing p130-angiomotin contained more stress fibres compared to cells expressing p80-angiomotin, resulting in altered cell shape and an approximately two-fold increase in cell-area. This is dependent on Rho activated kinase activity as addition of the ROCK inhibitor Y27632 prevented stress fibre formation (Worthylake and Burridge, 2003, J. Biol. Chem., 278:13578-13584; Nobes and Hall, 1999, J. Cell Biol., 144:1235-1244) (data not shown). Staining of MAE cells transfected with the N-terminal domain of p130-angiomotin showed a uniform co-localization with F-actin as opposed to the punctuated pattern observed with cells retrofected with p130-angiomotin plasmid. Expression of the N-terminal fragment resulted in similar accumulation of stress fibres which resulted in increased cell shape as seen with p130-angiomotin. This shows that the N-terminal domain contains an actin targeting sequence that mediates stabilization of stress fibres in the transfected cells. The staining of both p130 and the N-terminal fragment remained associated with phalloidin staining after treatment with cytochalasin D, an actin depolymerising agent, further indicating that the N-terminal domain mediate the targeting of p130-angiomotin to actin. The punctuated membrane localization of p130-angiomotin demonstrates that the C-terminal region, corresponding to p80-angiomotin, organizes p130-angiomotin in compartments along the stress fibres. Furthermore, the C-terminal concentrates p130-angiomotin to the central part of the cell whereas expression of the N-terminus alone is localized over the whole cell.

The different intercellular expression pattern of p80 and p130 in mouse aortic endothelial cells also argues that the isoforms have distinct cellular functions. We have previously shown that MAE-cells over-expressing p80-angiomotin have a 100% higher migration rate in response to chemotactic factors (Troyanovsky et al., 2001, J. Cell Biol., 152:1247-1254). Furthermore, p80 expression promotes tumour growth and invasion into surrounding muscle tissue in vivo (Levchenko et al., 2004, Oncogene, 23:1469-1473). A role of angiomotin in cell migration is further emphasized by the findings that angiomotin deficient mice die in utero at embryonic day 7-8 due to a migration defect in the anterior visceral endoderm (Shimono and Behringer, 2003, Curr. Biol., 13:613-7). Expression of p130 angiomotin in MAE cells did not, however, significantly affect the migration rate as analyzed in the Boyden chamber or the wound healing assays. Both the p130- and p80-angiomotin isoforms contain an angiostatin binding domain that is not present in the other two members of the angiomotin protein family (Bratt et al., 2002, Gene, 298:69-77). We show here that the angiostatin binding domain of p130-angiomotin is, similar to that of p80-angiomotin, exposed at the cell surface. Addition of angiostatin to MAE cells transfected with p130-angiomotin inhibited cell migration at similar efficacy to that of p80 expressing cells. Angiostatin did however not affect the stress fibre formation or the cell shape of p130-angiomotin expressing cells. These data argue that angiostatin primarily interferes with potential signalling pathways elicited by the p80 domain.

The distribution of p130-angiomotin during embryogenesis and in adult tissues indicates that its expression is tight regulated; p130-angiomotin is only found in the thymus, testis and placenta, whereas its shorter isoform is more widely distributed. In the placenta, angiomotin is expressed by endothelial cells and cytotrophoblasts. The latter cells are characterized by their ability to invade the deciduas and replace the endothelial lining of uterial arterioles, and to a lesser extent veins. This important process establishes the utero-placental circulation (reviewed in Bratt et al., 2002, Gene, 298:69-77). Interestingly, invasive cytotrophoblasts acquire endothelial characteristics and express genes otherwise specific for endothelium (e.g. PECAME and VE-cadherin). Endothelial cells and human cytotrophoblasts are so far the only primary cells that have been shown to express both isoforms of angiomotin. Consequently, both cell types also respond to angiostatin by inhibition of migration or invasion.

The formation of a vascular tube includes the inductive signalling from surrounding tissue that induces the directed migration of cells (reviewed in (Cammeliet, 2000, Nature Med., 6:389-395). Tip cells sense the chemotactic gradient by the extension of filopodia (Gerhardt et al., 2003, J. Cell Biol., 161:1163-1177). Tube formation also involves the re-shaping of the cytoskeleton of cells to form a closed monolayer, which surrounds a hollow fluid-filled lumen. The formation of cell junctions seals the endothelial layer to prevent para-cellular leakage and mark the border between apical and basal membrane domains (reviewed in Bazzoni and Dejana, 2004, Physiol. Rev., 84:869-901). Here we show that endothelial cells express two isoforms of angiomotin, the p80 and p130.

We speculate that these isoforms play distinct roles during angiogenesis where p80-angiomotin promotes cell migration and p130-angiomotin is involved in changing the morphology of endothelial cells during tubulogenesis.

We provide evidence that both p80- and p130-angiomotin are integrated membrane proteins. We further show that both isoforms co-localize with ZO-1 in endothelial cells in vitro and in vivo. The data indicate that angiomotin is a novel component of endothelial tight junction protein complex and may play a functional role in the assembly of endothelial cell-cell junctions.

We provide the following evidence for the model of angiomotin as a membrane integrated protein:
1. Angiomotin was biotinylated by cell-surface labelling of intact transfected as well as primary endothelial cells.
2. Angiomotin epitopes are sensitive to trypsin degradation.
3. Angiostatin binds specifically to the surface of angiomotin-transfected cells.
4. Antibodies directed against the angiostatin binding domain bind to angiomotin on the cell surface of transfected cells. We conclude that angiomotin is localised on the cell surface where it can serve as a receptor for angiostatin. Antibody mapping suggest a transmembrane model for angiomotin where the hydrophobic regions flanking the angiostatin binding domain can form two transmembrane helices leaving the central part of the angiostatin binding domain exposed on the cell surface.

Angiomotin lacks a signal peptide as judged the sequence analysis which indicates that it does not reach the membrane by the classical secretory pathway, which begins with incorporation of the peptide into the membrane of the endoplasmic reticulum (ER) during protein synthesis (Keenan et al., 2001, Ann. Rev. Biochem., 70:755-775). However, proteins may insert into the membrane, or can be secreted, by other mechanisms (Nickel, 2003, Eur. J. Biochem., 270:2109-2119). Thus, proteins can be secreted through what is usually referred to as non-classical secretion. Examples of such proteins are bFGF (Prudovsky et al., 2002, J. Cell Biol., 158:201-8), thioredoxin (Tanudji et al., 2003, Am. J. Physiol. Cell Physiol., 284:C1272-1279), and TSAP6 (Amzallag et al., 2004, J. Biol. Chem., 279:46104-46112). There are also examples of membrane proteins that lack signal peptides and are thus not inserted into the ER during protein synthesis. One group of membrane proteins, which is usually referred to as tail-anchored proteins, for example synaptobrevin, are post-translationally inserted into the membrane (Whitley et al., 1996, J. Biol. Chem., 271:7583-7586). The model of angiomotin proposed by us, however, argues against angiomotin belonging to this class of membrane proteins. Furthermore, the protein HASPB expressed by the parasite Leishmania in eukaryotic cells can also insert into the inner leaflet of the plasma membrane from a cytoplasmic location and then transfer across the membrane (Denny et al., 2000, J. Biol. Chem., 275:11017-11025). Annexin, which interestingly also binds to angiostatin (Tuszynski et al., 2002, Microvasc. Res., 64:448-462), is a membrane bound protein which can be inserted into the membrane and assume a membrane spanning conformation by a mechanism which involves the transition from a membrane-bound state to a membrane-spanning state (Langen et al., 1998, Proc. Natl. Acad. Sci. USA., 95:14060-14065). Our data suggest that angiomotin has properties similar to annexin and HASPB in that, while lacking a signal peptide, still can assume a membrane-spanning conformation by insertion of the hydrophobic helices flanking the angiostatin binding domain into the membrane.

Previous data show that angiomotin expression correlates with increased cell motility and that this effect is blocked by angiostatin, suggesting that angiostatin inhibits angiogenesis by blocking angiomotin-induced cell motility (Troyanovsky et al., 2001, J. Cell Biol., 152:1247-1254; Levchenko et al., 2003, J. Cell Sci., 116:3803-3810; Levchenko et al., 2004, Oncogene, 23:1469-1473; Shimono and Behringer, 2003, Curr. Biol., 13:613-7). This study shows that angiomotin, in addition to controlling cell motility, (Folkman, 1995, Nature Med., 1:27-31) localizes to cell-cell contacts in vivo and in vitro and, when transfected into CHO cells, (Hanahan and Folkman, 1996, Cell, 86:353-364) co-localize with the TJ protein ZO-1, (O'Reilly et al., 1994, Cell, 79:315-328) recruits ZO-1 to stress fibers, (Ji et al., 1998, Faseb J., 12:1731-8) control permeability and (Claesson-Welsh et al., 1998, Proc. Natl. Acad. Sci. USA, 95:5579-5583) bind to and co-localize with the TJ protein MAGI-1. We conclude that angiomotin in a TJ protein. Previously, we have reported that transgenic mice expressing dominant negative angiomotin in endothelial cells examined at embryonic day 9.5 displayed leaky blood vessels leading to severe bleeding in the brain (Levchenko et al., 2003, J. Cell Sci., 116:3803-3810), findings which are consistent with a role for angiomotin in decreasing permeability of TJs.

During angiogenesis cells migrate to the site of new vessels, and then mature into a functional vessel with mature tight junctions and adherence junctions. Both migration and maturation are crucial steps during angiogenesis and our data, together with previous findings, suggest that angiomotin can control both. In a similar manner, JAM-1, a transmembrane TJ protein, not only reduces para-cellular permeability (Tanudji et al., 2003, Am. J. Physiol. Cell Physiol., 284:C1272-9) but is also required for bFGF-induced motility of endothelial cells (Amzallag et al., 2004, J. Biol. Chem., 279:46104-46112; Whitley et al., 1996, J. Biol. Chem., 271:7583-7586). In this study, angiostatin specifically affected angiomotin-induced motility but not angiomotin-controlled permeability. This indicates that angiostatin exclusively affects motile endothelial cells participating in angiogenesis and not endothelial cells in established vessels. In addition, angiostatin has not been reported to increase vessel permeability in clinical trials (Denny et al., 2000, J. Biol. Chem., 275:11017-11025).

How does angiomotin control permeability? The extra-cellular domain of angiomotin could participate in homotypic binding and act as a seal in TJs, much like occluding or claudin. Another possibility is that the effect of angiomotin is secondary and the result of signalling and/or recruitment of other proteins to TJs. Angiomotin like 1/JEAP is also reported to localize to TJs (Dejana, 2004, Nat. Rev. Mol. Cell. Biol., 5:261-270) but since this protein as well as protein family member angiomotin-like 2 lacks an angiostatin binding domain (Bratt et al., 2002, Gene, 298:69-77) we predict that angiomotin exclusively is the mediator of the effect of angiostatin.

We have shown that p130-, but not p80-angiomotin interacts with the TJ protein MAGI-1b which shows that the N-terminal extension domain of 409 residues of p130 angiomotin is necessary for this interaction. Although we have not shown a direct interaction between p130-angiomotin and MAGI-1 it is interesting to note that the N-terminus of p130 contains several praline-rich domains, including a PpxY motif, that could serve as bindings motifs for the WW domains of MAGI-1 (Dobrosotskaya and James, 2000, Biochem. Biophys. Res. Comm., 270:903-9; Kay et al., 2000, Faseb J., 14:231-241). However, the interaction with MAGI-1 is not necessary to target angiomotin to TJs as angiomotin by itself localizes to cell-cell contacts and controls permeability.

Cell motility and TJs are both closely regulated by the actin cytoskeleton and the Rho family of GTPases. For example, while both Rho and Rac are recognized as two of the most important regulators of cell motility (Burridge and Wennerberg, 2004, Cell, 116:167-179) both are crucial in regulating TJ formation downstream of Par-3 and Par-6, respectively (Chen and Macara, 2005, Nat. Cell Biol., 7:262-9; Ozdamar et al., 2005, Science, 307:1603-1609; Patrie et al., 2002, J. Biol. Chem., 277:30183-30190). It is possible that angiomotin regulates both permeability and motility by influencing the actin cytoskeleton. Actually, there is evidence that angiostatin and angiomotin are linked to Rho signalling (Ernkvist et al., 2005, submitted; Gupta et al., 2001, EMBO Rep., 2:536-540) (Bratt and Holmgren), and it is tempting to speculate that control of the actin cytoskeleton is at the heart of both properties of angiomotin. In line with this, MAGI-1 binds actin binding proteins alfa-actinin 4 and synaptopodin (Patrie et al., 2002, J. Biol. Chem., 277:30183-30190).

The encouraging results achieved with VEGF antibody bevacizumab (Hurwitz et al., 2004, N. Engl. J. Med., 350:2335-42) show that we are entering the era of anti-angiogenic therapy, but also indicate that additional angiogenesis inhibitors are needed. Our results provide the rationale for designing antibodies that bind the angiostatin binding domain of angiomotin. It has been shown that anti-angiogenic antibodies that affect cell-cell interactions by blocking VE-cadherin may be designed as not to effect permeability of mature vessels but only block neovascularisation (Corada et al., 2002, Blood, 100:905-11). This study suggests that an angiostatin mimetic antibody may block angiogenesis by exclusively controlling cell migration without affecting the cell-cell contacts of endothelial cells in mature vessels

### EXAMPLE 2

### Preferred Pharmaceutical Formulations and Modes and Doses of Administration

The polypeptides, polynucleotides and antibodies of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The polypeptides, polynucleotides and antibodies of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for administration. A device which delivers a pulsed electric field to cells increases the permeability of the cell membranes to the drug, resulting in a significant enhancement of intracellular drug delivery.

Polypeptides, polynucleotides and antibodies of the invention can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of administration is the ReGel injectable system that is thermosensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Polypeptides, polynucleotides and antibodies of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient (Derossi et al., 1998, Trends Cell Biol., 8, 84-87.

Preferably, the pharmaceutical formulation of the present invention is a unit dosage containing a daily dose or unit, daily sub-dose or an appropriate fraction thereof, of the active ingredient.

The polypeptides, polynucleotides and antibodies of the invention can be administered by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the polypeptides, polynucleotides and antibodies of the invention can be administered alone but will generally be administered in admixture with a suitable pharmaceutical excipient diluent or carrier selected with regard to the intended route of administration and standard pharmaceutical practice.

The polypeptides, polynucleotides and antibodies of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, oral or parenteral administration of the polypeptides, polynucleotides and antibodies of the invention is the preferred route, being the most convenient.

For veterinary use, the polypeptides, polynucleotides and antibodies of the invention are administered as a suitably acceptable formulation in accordance with normal veterinary practice and the veterinary surgeon will determine the dosing regimen and route of administration which will be most appropriate for a particular animal.

The formulations of the pharmaceutical compositions of the invention may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Preferred unit dosage formulations are those containing a daily dose or unit, daily sub-dose or an appropriate fraction thereof, of an active ingredient.

A preferred delivery system of the invention may comprise a hydrogel impregnated with a polypeptides, polynucleotides and antibodies of the invention, which is preferably carried on a tampon which can be inserted into the cervix and withdrawn once an appropriate cervical ripening or other desirable affect on the female reproductive system has been produced.

It should be understood that in addition to the ingredients particularly mentioned above the formulations of this invention may include other agents conventional in the art having regard to the type of formulation in question.

### EXAMPLE 3

### Exemplary Pharmaceutical Formulations

Whilst it is possible for a polypeptides, polynucleotides and antibodies of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a polypeptides, polynucleotides and/or antibody of the invention.

### EXAMPLE 3A

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### EXAMPLE 3B

### Capsule Formulations

### Formulation A

A capsule formulation is prepared by admixing the ingredients of Formulation D in Example C above and filling into a two-part hard gelatin capsule. Formulation B (infra) is prepared in a similar manner.

Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

### Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### EXAMPLE 3C

### Injectable Formulation

| | | |
|---|---|---|
| Active ingredient | 0.200 | g |
| Sterile, pyrogen free phosphate buffer (pH7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### EXAMPLE 3D

### Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### EXAMPLE 3E

### Syrup Suspension

| | | |
|---|---|---|
| Active ingredient | 0.2500 | g |
| Sorbitol Solution | 1.5000 | g |
| Glycerol | 2.0000 | g |
| Dispersible Cellulose | 0.0750 | g |
| Sodium Benzoate | 0.0050 | g |
| Flavour, Peach 17.42.3169 | 0.0125 | ml |
| Purified Water q.s. to | 5.0000 | ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### EXAMPLE 3F

### Suppository

| | mg/suppository |
|---|---|
| Active ingredient (63 μm)* | 250 |
| Hard Fat, BP (Witepsol H15-Dynamit Nobel) | 1770 |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE 3G

### Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE 3H

### Creams and Ointments

Described in Remington, The Science and Practise of Pharmacy, 19^{th} ed., The Philadelphia College of Pharmacy and Science, ISBN 0-912734-04-3.

### EXAMPLE 3I

### Microsphere Formulations

The compounds of the invention may also be delivered using microsphere formulations, such as those described in Cleland (1997, Pharm. Biotechnol. 10:1-43; and 2001, J. Control. Release 72:13-24).

1. An isolated or recombinant p130-angiomotin polypeptide for modulating angiogenesis and/or tumour formation.

2. The polypeptide of claim 1 wherein the polypeptide is an isolated or recombinant mammalian p130-angiomotin.

3. The polypeptide of claim 2 wherein the polypeptide is an isolated or recombinant human p130-angiomotin.

4. The polypeptide of any one of claims 1 to 3 wherein the polypeptide comprises:
(i) the sequence of SEQ ID NO:1; or
(ii) a sequence which has at least 80% and/or at least 90% and/or at least 95% and/or at least 98% identity to SEQ ID NO:1 and provides a functional polypeptide; or
(iii) a functional fragment of SEQ ID NO:1 or the sequence of (ii).
and wherein the polypeptide is not p80-angiomotin or a fragment of p80-angiomotin.

5. The polypeptide of claim 4 wherein the functional fragment of SEQ ID NO:1 consists of amino acid residues 1 to 409 of SEQ ID NO:1.

6. An antibody or a fragment thereof which is capable of binding to a p130-angiomotin polypeptide as defined in any one of claims 1 to 5.

7. The antibody or fragment of claim 6 which is capable of binding to a region of a p130-angiomotin polypeptide defined by residue 1 to residue 409 of SEQ ID NO:1 but that is not capable of binding to a p80-angiomotin polypeptide.

8. The antibody or fragment of claim 6 which capable of binding to a region of a p130-angiomotin polypeptide defined by residue 410 to residue 1084 of SEQ ID NO:1 but that is not capable of binding to a p80-angiomotin polypeptide.

9. The antibody or fragment of claim 6 which is capable of binding to a region of a p130-angiomotin polypeptide defined by residue 871 to residue 1013 of SEQ ID NO:1 but that is not capable of binding to a p80-angiomotin polypeptide.

10. Use of an angiomotin polypeptide as defined in any one of claims 1 to 5 and/or its encoding polynucleotide (and/or antisense polynucleotide) for generating antibodies capable of binding to a p110-angiomotin polypeptide as defined in any one of claims 1 to 5.

11. An angiomotin polypeptide as defined in any one of claims 1 to 5 and/or its encoding polynucleotide (and/or antisense polynucleotide) and/or an antibody or fragment as defined in any one of claims 6 to 9 for use in medicine.

12. Use of an angiomotin polypeptide as defined in any one of claims 1 to 5 and/or its encoding polynucleotide (and/or antisense polynucleotide) and/or an antibody or fragment as defined in any one of claims 6 to 9, in the manufacture of a medicament for modulating angiogenesis and/or tumour formation.

13. The use of claim 12 wherein the medicament prevents and/or reduces angiogenesis and/or tumour formation.

14. Use of an angiomotin polypeptide as defined in any one of claims 1 to 5 and/or its encoding polynucleotide (and/or antisense polynucleotide) and/or an antibody or fragment as defined in any one of claims 6 to 9, in the manufacture of a medicament for treating a subject with an angiogenesis-related disease or disorder.

15. Use of an angiomotin polypeptide as defined in any one of claims 1 to 5 and/or its encoding polynucleotide (and/or antisense polynucleotide) and/or an antibody or fragment as defined in any one of claims 6 to 9 in the manufacture of a vaccine for vaccinating a subject with, or at risk of, angiogenesis and/or tumour formation and/or an angiogenesis-related disease or disorder.

16. The use of claim 14 or 15 wherein the angiogenesis-related disease or disorder is cancer, a solid tumour, haemangioma, ocular neovascularisation, diabetic retinothapy, macular degeneration, rheumatoid arthritis, inflammatory conditions, psoriasis, chronic inflammation of the intestines, asthma or endometriosis.

17. Use of an antibody or fragment as defined in any one of claims 6 to 9 in the detection and/or measurement of angiogenesis and/or tumour formation in a test sample.

18. A pharmaceutical composition for modulating angiogenesis and/or tumour formation, comprising an effective amount of an angiomotin polypeptide as defined in any one of claims 1 to 5 and/or its encoding polynucleotide (and/or antisense polynucleotide) and/or an antibody or fragment as defined in any one of claims 6 to 9, and a pharmaceutical excipient or diluent.

19. The pharmaceutical composition of claim 18 which prevents and/or reduces angiogenesis and/or tumour formation.

20. A vaccine for modulating angiogenesis and/or tumour formation, comprising an effective amount of an angiomotin polypeptide as defined in any one of claims 1 to 5 and/or its encoding polynucleotide (and/or antisense polynucleotide), and an excipient or diluent.

21. The vaccine of claim 20 or pharmaceutical composition of claim 18 or 19 further comprising at least one additive for assisting or augmenting the action of the polypeptide and/or polynucleotide (and/or antisense polynucleotide) and/or antibody or fragment therein.

22. The vaccine of claim 21 or the pharmaceutical composition of claim 21 wherein the at least one additive is an immunostimulatory molecule.

23. The vaccine of claim 22 or the pharmaceutical composition of claim 22 wherein the immunostimulatory molecule is a cytokine or polynucleotide (and/or antisense polynucleotide) encoding a cytokine.

24. The vaccine of any one of claims 20 to 23 wherein the vaccine comprises a cell or cell extract.

25. The vaccine of claim 24 wherein the cell is an antigen presenting cell which is loaded with the angiomotin polypeptide or transfected with the encoding polynucleotide (and/or antisense polynucleotide).

26. The vaccine of claim 25 wherein the cell is a tumour cell expressing angiomotin or an endothelial cell expressing angiomotin.

27. A method of generating an immune response against angiomotin in a mammal, the method comprising the steps of:
(iii) stimulating ex vivo immune cells collected from the mammal with an angiomotin polypeptide as defined in any one of claims 1 to 5 or its encoding polynucleotide (and/or antisense polynucleotide);
(iv) transferring the stimulated immune cells back into the mammal, such that transfer of the cells back into the mammal generates and immune response against angiomotin.

28. The method of claim 27 wherein the mammal is a human.

29. The method of claim 27 or 28 wherein the immune response serves prophylactically or therapeutically to inhibit the onset or progress of an angiogenesis-related disease.

30. The use of any one of claims 10 to 17 or the pharmaceutical composition of any one of claims 18, 19 or 21 or the vaccine of any one of claims 20 to 26 or the method of any one of claims 27 to 29 wherein the encoding polynucleotide comprises:
(i) the polynucleotide (and/or antisense polynucleotide) of SEQ ID NO:2; or
(ii) a polynucleotide (and/or antisense polynucleotide) which has at least 80% and/or at least 90% and/or at least 95% and/or at least 98% identity to SEQ ID NO:2 and/or is capable of hybridising to SEQ ID NO:2 under conditions of 2×SSC at 65° C. and/or which encodes a functional polypeptide; or
(iii) a fragment of SEQ ID NO:2 which encodes a functional polypeptide
and wherein the polynucleotide (and/or antisense polynucleotide) does not encode a p80-angiomotin or a fragment of p80-angiomotin.
